(19) United States
(12) Patent Application Publication
(10) Pub. No.: US 2024/0083971 A1
(43) Pub. Date: Mar. 14, 2024

(54) IMMUNE EFFECTOR CELL COVALENT IMMUNE RECRUITING (CIR) MOLECULES AND METHODS AND USES THEREOF

(71) Applicant: McMaster University, Hamilton (CA)

(72) Inventors: Anthony Rullo, Hamilton (CA); Yonghong Wan, Ancaster (CA); Eden Kapcan, Hamilton (CA); Zi Ling Yang, Waterloo (CA)

(21) Appl. No.: 18/234,216

(22) Filed: Aug. 15, 2023

Related U.S. Application Data
(60) Provisional application No. 63/398,427, filed on Aug. 16, 2022.

(51) Int. Cl. C07K 14/735; A61P 35/00; C12N 5/078

(52) U.S. Cl. CPC C07K 14/70535; A61P 35/00; C12N 5/0634; C12N 2510/00

(57) ABSTRACT

The present description relates to covalent immune recruiters (CIRs) comprising an Fc receptor targeting domain (FTD), a covalent binding group (CBG), and a target binding domain (TBD), wherein on binding of the FTD to a cognate Fc receptor, the CBG forms a covalent bond with an amino acid in the Fc receptor. The present description also includes functionalized cells modified by covalent binding of the CIR, methods and uses thereof, for example, methods of functionalizing cells, and methods and uses of such cells for recognition of target cells.

### CROSS-REFERENCE TO RELATED APPLICATIONS

This application claims the benefit of priority to U.S. Provisional Application No. 63/398,427, filed Aug. 16, 2022, the contents of which are incorporated herein by reference in their entirety.

### INCORPORATION OF SEQUENCE LISTING

A computer readable form of the Sequence Listing “3244-P69131US01_SequenceListing.xml” (35,941 bytes) created on Aug. 15, 2023, is herein incorporated by reference.

### FIELD

The present disclosure relates to covalent immune recruiters (CIRs) comprising an Fc receptor targeting domain, a covalent binding group, and a target binding domain, as well as functionalized cells comprising said target binding domain, and methods of using such CIRs and functionalized cells.

### BACKGROUND

Immune Fc receptors (FcRs) like CD16 and CD64 are involved in activation of anti-tumor immune responses. Molecules such as synthetic antibody mimics (SyAMs) and antibody recruiting molecules (ARMs) which target FcRs are currently in pre-clinical studies. SyAMs, and ARMs are limited by reversible weak binding affinity for FcRs which attenuates their anti-tumor function. SyAMs and ARMs are also rapidly cleared from systemic circulation. Additionally, ARMs are dependent on natural antibodies being present in human serum of sufficient abundance and affinity. mAbs are also intrinsically limited by their weak affinity for FcRs coupled with the general limitations of protein therapies.

In the specific context of activation of endogenous macrophages, no bi-specific protein “engager” therapies exist, in contrast to analogous therapeutics that recruit endogenous NK cells and T cells against tumors (i.e. bi-specific T cell engagers (BiKEs) and bi-specific NK cell engagers (BiTEs)). Macrophages represent strategic immune cells for immunotherapy due to their abundance in the tumor environment and antigen presentation. This growing interest in macrophage recruitment is exemplified by the recent development of CAR-macrophages.

U.S. patent application Ser. No. 17/249,332 describes molecular adapters, termed covalent immune recruiters (CIRs), that chemically link a targeting domain to an antibody molecule such as an IgG, which targeted antibody can be used to direct immune cells to target domains on cancer cells.

### SUMMARY

CIRs represent a unique small molecule synthetic engager strategy to redirect macrophage function selectively against tumor cells. Bi-specific protein engager therapies that redirect NK cell function via CD16 FcR binding are intrinsically limited by their rapid clearance from systemic circulation which can also be addressed using CIRs against CD16a. Additionally, although BiKEs can bind CD16a with high (nM) affinity, they cannot easily be designed to dramatically enhance their binding affinity for CD16a in contrast to CIR covalent binding. The immune cell CIRs described herein bind covalently to immune FcRs like CD16 and CD64 involved in activation of an anti-tumor immune response. This enables the molecule to a) enhance the recruitment and activation of natural endogenous immune effector cells like macrophages, against cancers of interest and b) retain in the circulation for much longer periods of time which reduces dosage requirements/times and increases pharmacodynamic control.

Described herein is the development of covalent immune recruiters (CIRs) that can be used to covalently modify an immune cell such as an FcR-expressing monocyte, M1M2 macrophage, or tumor-associated macrophage (TAM) to exert control over immune recognition. The resulting functionalized cells are able to affect immune recognition of model targets including tumor proteins on human cells, and induce a cytotoxic and/or phagocytic immune response targeting cells expressing the target tumor protein. Accordingly, functionalized cells comprising an endogenous Fc receptor modified by a CIR described herein are useful tools for triggering phagocytosis of, or a cytotoxic response to, target cells. Also described herein are precursor compounds and methods for generating the CIRs described herein, comprising contacting an FcR targeting component comprising an FcR targeting domain, a covalent binding group, and an acceptor group for covalent attachment of the TBD (for example in vitro or directly in vivo), and a target binding component comprising a TBD and a cognate donor group (i.e. “click handle”) for covalent attachment to the acceptor group of the FcR targeting component, wherein the FcR targeting component and the target binding component are contacted under suitable conditions to allow binding and covalent attachment of the acceptor group and cognate donor group, thereby generating a CIR.

An aspect includes a covalent immune recruiter (CIR) comprising an Fc receptor (FcR) targeting domain (FTD), a covalent binding group (CBG), and a first target binding domain (TBD), wherein the FTD specifically binds an FcR on an immune cell, and wherein the CBG comprises a functional group that, on binding of the FTD to the FcR, forms a covalent bond with an amino acid in the FcR.

In an embodiment, the FcR is an FcγR, optionally selected from CD64, CD32, CD16a, and CD16b, optionally human CD64.

In an embodiment, the FTD comprises a peptide having the sequence of SEQ ID NO: 1 or a functional variant thereof or SEQ ID NO: 2 or a functional variant thereof. In an embodiment, the FTD comprises a peptide having the sequence SEQ ID NO: 3, SEQ ID NO: 4, SEQ ID NO: 7, SEQ ID NO: 8, SEQ ID NO: 9, SEQ ID NO: 10, SEQ ID NO: 11, or SEQ ID NO: 12, or functional variants thereof. In an embodiment, the peptide is C-terminally amidated. In an embodiment, the peptide is not C-terminally amidated.

In an embodiment, the TBD binds a protein that is overexpressed in a disease, disorder or condition, optionally cancer.

In an embodiment, the TBD comprises a prostate specific membrane antigen (PSMA) binding ligand. In an embodiment, the PSMA binding ligand comprises glutamate urea lysine (GUL), or a functional variant thereof.

In an embodiment, the TBD comprises a uPAR binding ligand. In an embodiment, the uPAR binding ligand comprises a synthetic uPAR binding peptide ligand, optionally comprising a sequence of SEQ ID NO: 15 or a functional variant thereof, or SEQ ID NO: 16 or a functional variant thereof. In an embodiment, the uPAR binding peptide ligand comprises a sequence of SEQ ID NO: 16, or a functional variant thereof.

In an embodiment, the TBD comprises biotin.

In an embodiment, the CBG comprises SuFEx, optionally fluorosulfonate, fluorosulfate, or sulfonyl fluoride.

In an embodiment, the CIR further comprises a second TBD, optionally comprising a third TBD.

An aspect includes a functionalized cell modified by a CIR described herein.

In an embodiment, the cell is selected from lymphocytes, monocytes, macrophages, polymorphonuclear cells, erythrocytes and megakaryocytes. In an embodiment, the cell is a tumor-associated macrophage.

In an embodiment, the cell is selected from B cells, NK cells, macrophages, neutrophils, basophils, eosinophils, and dendritic cells, optionally tumor-associated macrophages.

An aspect includes a method of generating a functionalized cell, the method comprising: providing a cell comprising an FcR; and contacting the cell with a CIR described herein.

An aspect includes a functionalized cell generated using the method of providing a cell comprising an FcR; and contacting the cell with a CIR described herein.

An aspect includes a method of treating or preventing a disease, disorder or condition that is treatable or preventable by immunotherapy, comprising administering a therapeutically effective amount of a) a CIR described herein; or b) a functionalized cell described herein; to a subject in need thereof.

In an embodiment, the disease, disorder, or condition that is treatable by immunotherapy is a cancer, an autoimmune disease, allergy, or transplant rejection.

In an embodiment, the cells are autologous.

In an embodiment, the cells are allogenic.

A further aspect includes a kit comprising: a) an FcR targeting component comprising an FcR targeting domain, a covalent binding group, and an acceptor group for covalent attachment of a target binding domain and/or a target binding component comprising a target binding domain and a cognate donor group for covalent attachment to the acceptor group of the FcR targeting component; b) a CIR described herein; or c) a functionalized cell described herein; and optionally one or more of a suitable container or packaging therefor and/or instructions for use thereof.

Other features and advantages of the present description will become apparent from the following detailed description. It should be understood, however, that the detailed description and the specific examples, while indicating embodiments of the disclosure, are given by way of illustration only and the scope of the claims should not be limited by these embodiments but should be given the broadest interpretation consistent with the description as a whole.

### BRIEF DESCRIPTION OF DRAWINGS

The embodiments of the present disclosure will now be described in greater detail with reference to the attached drawings in which:

FIG. 1 shows a schematic illustration of i) an antibody CIR (cARM) (prior art) vs. ii) an immune cell CIR (described herein).

FIG. 2 shows computational docking of CP33 peptide to the IgG binding site of the CD64 macrophage receptor. The position of the side chains of the proximal reactive amino acids on CD64 to CP33 peptide are shown. The N terminus of CP33 was selected as the initial point of covalent chemistry incorporation to enable covalent binding of the peptide to CD64 via linkage to lysine 173 or tyrosine 176. SuFEX chemistry was initially chosen as the covalent binding chemistry of interest. SuFEx can be installed by incorporating a tyrosine amino acid at any position of interest on CP33, for example a C-terminal tyrosine (SEQ ID NO: 17).

FIG. 3 shows a schematic of the general design of macrophage (CD64) targeting CIRs against PSMA on prostate cancer cells. A strained alkyne is used for “click chemistry” connection of a tumor binding molecule to an FcR binding peptide. The CP33 C-term tyrosine mutant peptide is shown on the left. The PSMA binding ligand with linker and dibenzocyclooctyne (DBCO) is shown on the right.

FIG. 4A-4C shows biolayer interferometry (BLI) binding data of native CP-33-linker-biotin conjugate to CD64. FIG. 4A shows non-linear curve fitting analysis used to extract a dissociation constant for binding=10⁻⁸ M. FIG. 4B shows the structure of azido-modified native CP-33. FIG. 4C shows the structure of DBCO biotin.

FIG. 5 shows BLI binding data of native CP-33-linker-biotin conjugate to human CD64 with high selectivity vs related immune receptors such as human CD16a and mouse CD64.

FIG. 6A-6B depicts results for the CP-33(G13Y mutant). FIG. 6A shows BLI binding data of CP-33(G13Y mutant)-linker-biotin conjugate to human CD64 indicating significant perturbation of CD64 binding. FIG. 6B shows the structure of the CP-33(G13Y mutant). (Note: the linker-biotin is attached to the azide; DBCO-Biotin not shown).

FIG. 7A-7C depicts results for the CP-33(C-term “Y” insertion mutant). FIG. 7A shows BLI binding data of CP-33(C-term “Y” insertion mutant)-linker-biotin conjugate to human CD64. FIG. 7B shows the structure of the CP-33(C-term “Y” insertion mutant). (Note: the linker-biotin is attached to the azide; DBCO-Biotin not shown). A higher binding affinity for CD64 but lower binding amplitude is calculated/observed. FIG. 7C shows BLI binding data of the CP-33(C-term “Y” insertion mutant) to human CD64 with high selectivity vs related immune receptors such as human CD16a and mouse CD64. High selectivity for human CD64 is observed.

FIG. 8A-8B depicts results for the CP-33(N-term “Y” insertion mutant). FIG. 8A shows BLI binding data of CP-33(N-term “Y” insertion mutant)-linker-biotin conjugate to human CD64. FIG. 8B shows the structure of the CP-33(N-term “Y” insertion mutant). (Note: the linker-biotin is attached to the azide; DBCO-Biotin not shown). A comparable binding affinity for CD64 (and binding amplitude) is observed compared to native CP33.

FIG. 9A-9C shows chemical structures of synthetic intermediates for a key lead CIR fragment: CP33-Nterm-SuFEx-linker-fluorescein for fluorescence SDS-PAGE kinetics studies described herein. FIG. 9A shows the structure of CP33-Nterm-SuFEx intermediate (Note: the linker-fluorescein is attached to the azide. FIG. 9B shows the structure of DBCO-fluorescein and FIG. 9C shows the structure of Fmoc-Tyr-OSuFEx-COOH.

FIG. 10 shows SDS-PAGE analysis of covalent binding of CIR fragment:CP33-Nterm-SuFEx-linker-fluorescein with human CD64 vs related off-target proteins CD16 and mouse CD64. Top. a fluorescence band (right panel) is only observed when CIR fragment is incubated with the target protein of interest human CD64. Left: Coomassie stain for protein content and MW, Right: fluorescence panel. Bottom: Kinetic monitoring of CD64 covalent reaction with CIR fragment over time leading to an increase in CD64 band fluorescence.

FIG. 11A-11B shows BLI NCIR bridging assay. FIG. 11A: 50 nM of biotinylated huCD64 loaded onto streptavidin probes, associated with 100-12.5 nM NCIR (“mNCIR”) preincubated with 200 nM of PSMA. The Anti-PSMA IgG trace was used as a positive control. FIG. 11B: Chemical structure of DBCO-Peg7-GUL.

FIG. 12 shows dual color phagocytosis assay of streptavidin beads mediated by CIRs and SyAM-P8-Biotin. Left Panel: Beads and U937 monocytes Only. No phagocytosis (Q2) is shown here. Middle panel: Beads, U937 monocytes and a non covalent analogue of CIR (“SyAM-P8-Biotin”) is able to enhance phagocytosis. Right Panel: Beads, U937 monocytes and CIR (“MCIR”) is able to enhance phagocytosis.

FIG. 13 shows the chemical structure of Sulfonyl Fluoride CIR: CP33-Nterm-aryl SuFEx (SEQ ID NO: 10). The fluorescein moiety shown in FIG. 9B can be added using click chemistry.

FIG. 14 shows chemical structures and SDS-PAGE gels comparing labelling chemistries. This SDS-PAGE was carried out as described for FIG. 10, except this image was taken after a 3 hour incubation. The fluorescence of the right sulfonyl fluoride bands is higher than the left fluorosulfonate bands, indicating the sulfonyl fluoride is a faster labelling chemistry.

FIG. 15 shows chemical structures for two sulfonyl fluoride CIR variations: cP33 ct-aryl SuFEx (top; SEQ ID NO: 12) and cp33 int-aryl SuFEx (bottom; SEQ ID NO: 14). The chemical structure for cP33 nt-aryl SuFEx is shown in FIG. 13.

FIG. 16 shows SDS-PAGE analysis of sulfonyl fluoride CIR CD64 covalent labelling specificity comparing CIR labelling CD64, CD16, and BSA. Dark bands show A647 fluorescence as measured using the Cy5 channel and represent covalent CIR binding. For each sample, 1.6 μM CIR clicked to A647 fluorescent dye was incubated with 800 nM of protein for 7 hours.

FIG. 17A-17C shows SDS-PAGE analysis of sulfonyl fluoride CIR CD64 covalent labelling kinetics. Dark bands show A647 fluorescence as measured using the Cy5 channel and represent covalent CIR binding. 1.6 μM of CIR was incubated with 800 nM CD64 for different amounts of time before being run on a SDS-PAGE gel. FIG. 17A shows cp33 ct-aryl SuFex; FIG. 17B shows cp33 int-aryl SuFEx; and FIG. 17C shows cP33 nt-aryl SuFEx.

FIG. 18A-18C shows fraction bound vs time graphs to determine K_{obs} of CIR covalently labeling CD64. The fluorescence intensity of each band in FIG. 17A-17C was measured and then compared to the fluorescence intensity of the band at 100% fraction labelled (where the fluorescence intensity reaches a plateau) to make a fraction bound vs time graph. The curve of the graph was fitted to a one-phase association curve in GraphPad Prism 8 to find the K_{obs}. FIG. 18A shows cp33 ct-aryl SuFex; FIG. 18B shows cp33 int-aryl SuFEx; and FIG. 18C shows cP33 nt-aryl SuFEx.

FIG. 19 shows stability studies for cP33-ct-aryl SuFEx showing the UV trace from the LC-MS. The cP33-ct-aryl SuFEx peak (base peak) elutes at 3.27 minutes and is denoted with an arrow in the top panel. Degradation peaks are labelled with an arrow in the bottom panel.

FIG. 20 shows stability studies for cP33-int-aryl SuFEx showing the UV trace from the LC-MS. The cP33-int-aryl SuFEx peak (base peak) elutes at 3.13 minutes and is denoted with an arrow in the top panel. Degradation peaks are labelled with an arrow in the bottom panel.

FIG. 21 shows stability studies for cP33-nt-aryl SuFEx showing the UV trace from the LC-MS. The cP33-nt-aryl SuFEx peak (base peak) elutes at 3.35 minutes and is denoted with an arrow in the top panel.

FIG. 22 shows sulfonyl fluoride CIR variants binding to CD64 on fixed U937 cells. CIRs were clicked to an A647 fluorescent handle and the mean fluorescence intensity was measured 7 hours post-incubation of the CIRs with fixed U937 cells. cP33-ct-aryl SuFEx (CT), cP33-int-aryl SuFEx (INT) and cP33-nt-aryl SuFEx (NT) were incubated at increasing concentrations.

FIG. 23 shows controls for Sulfonyl fluoride CIR variants binding to CD64 on fixed U937 cells. Controls were assayed with cP33-nt-aryl SuFEx CIR (NT), a non-covalent analog, and cP33-nt-aryl SuFEx reacted with un-activated U937 cells.

FIG. 24 shows CIR labeling of CD64 on live U937 cells as detected using streptavidin-PE. CIR-biotin was incubated with U937 cells for 24 hours at 37° C. along with IFNγ. The cells were then washed and incubated with streptavidin-PE. The streptavidin binds tightly to the biotin on the CIR and the fluorescent PE signal was read. cP33-ct-aryl SuFEx (CT), cP33-int-aryl SuFEx (INT), cP33-nt-aryl SuFEx (NT), and a noncovalent analog were incubated at increasing concentrations.

FIG. 25 shows controls for CIR labeling of CD64 on live U937 cells as detected using streptavidin-PE.

FIG. 26 shows MALDI peak for unlabeled CD64.

FIG. 27 shows MALDI peak for 5.4 μM CD64 incubated with 50 μM CIR in PBS for 24 hours.

FIG. 28 shows dual-colour phagocytosis assays showing the percent phagocytosis of fluorescent streptavidin beads by IFNγ activated U937 cells incubated with the indicated concentration of CIR-biotin (black bars) or 640 nM CIR without biotin (light grey bar), or without CIR (white bar), or unactivated U937 cells incubated with 640 nM CIR-biotin (dark grey bar). **** indicates p<0.0001.

FIG. 29 shows dual-colour phagocytosis assays showing phagocytosis of streptavidin beads by IFNγ activated U937 cells following CIR-biotin treatment compared to an analogous biotinylated antibody control or no treatment. *** indicates p<0.0003.

FIG. 30 shows greater activation of a NF-kB-Luc2 reporter cell line by CIR compared to both the analogous non-covalent version (NIR) and an untreated control.

FIG. 31 shows BLI uPAR MCIR bridging assay. A streptavidin biosensor was functionalized with biotinylated huCD64 and dipped into pre-equilibrated solution of human uPAR and either uPAR MCIR (MCIR) or the analogous non-covalent version (nMCIR).

FIG. 32 shows BLI uPAR MCIR bridging assay. A streptavidin biosensor was functionalized with biotinylated human uPAR and dipped into pre-equilibrated solution of huCD64 and either uPAR MCIR (MCIR) or the analogous non-covalent version (nMCIR).

FIG. 33 shows dual-colour phagocytosis assays showing phagocytosis of streptavidin beads labelled with biotinylated human uPAR by IFNγ activated U937 cells incubated with the indicated concentrations of either uPAR MCIR (mCIR) or the non-covalent version (nMCIR) compared to untreated control (No Treatment).

### DETAILED DESCRIPTION

Covalent immune recruiting molecules (CIRs) that irreversibly bind to natural antibodies and tumor targets to mediate a targeted anti-tumor immunotherapeutic response have been described previously. Serum antibodies were used as a proof of concept immune molecule in this previous disclosure (FIG. 1, i). Described herein is the development of CIRs that enable for direct irreversible (covalent) binding of immune Fc receptors on effector cells like NK cells and monocytes/macrophages (FIG. 1, ii).

The covalent immune cell FcR CIRs described herein are designed to maximize immune cell recruitment and activation at the tumor cell site avoiding the need for intermediate antibodies to affect FcR responses like antibody-dependent cellular phagocytosis (ADCP) and antibody-dependent cell-mediated cytotoxicity (ADCC). Via their unique ability to covalently bind FcR on immune cells, the CIRs described herein are expected to both overcome the rapid clearance of small molecule therapeutics from the circulation, and also maximize the affinity for the limiting binding interaction which drives FcR clustering and activation. Published data looking at FcR mechanisms of tumor immunotherapeutic antibodies collectively suggest the ADCP/ADCC anti-tumor efficacy of immunotherapeutic antibodies and antibody recruiting molecules in “synthetic immunity” are limited by antibody affinity for FcR. In contrast, the immune cell CIR technology described herein employs established weak affinity but selective peptide agonists of human IgG1 binding to FcR (e.g. CD16a and CD64), or novel designer peptides, engineered with covalent chemistry that is only activated upon peptide:FcR binding. These covalent peptides are appended to established tumor antigen binding synthetic molecules, to afford a bi-functional synthetic tumor immunotherapeutic. This strategy is highly tumor target tunable given the availability of different tumor antigen binding molecules selective for, for example, PSMA on prostate cancer and tumor neovasculature, folate receptor alpha on ovarian, breast, and brain cancer cells, and urokinase plasminogen activator receptor (uPAR) widely expressed on a number of highly metastatic cancers. This strategy also provides high selectivity given the requirement for binding to multiple tumor antigens on a target tumor cell to activate immune cell FcR dependent responses like ADCP/ADCC. This is because FcRs must be both bound and clustered by a high number of therapeutic antibodies, or in the present case, several immune cell CIRs bound to several tumor antigens on a target cell simultaneously. The approach described herein further enables for facile multi-specific tumor targeting, with multiple TBDs covalently bound to the same FcR molecule simultaneously, each encoding a different tumor antigen selectivity, thereby generating a multivalent FcR. This can be achieved by designing CIRs which eject the FcR binding peptide upon covalent reaction with a proximal binding site nucleophilic amino acid, which will enable a subsequent CIR to bind and covalently label the same FcR. Alternatively or additionally, CIRs can be designed which incorporate multiple TBDs into the a single CIR. A wide range of different covalent chemistries can be incorporated into CIRs, each with different FcR labeling kinetics and binding site amino acid selectivities. These covalent chemistries include, without limitation, acylimidazole esters, sulfonyl fluoride exchange chemistries, in addition to a number of known proximity activated acylation and alkylation chemistries typically employed in the development of covalent drugs and enzyme inhibitors such as alpha haloketones, vinyl sulfones and sulfonamides, acrylamides, tosyl sulfones, boronic acids/esters, and carbonyl compounds. This chemistry can be efficiently installed within a small peptide scaffold on solid support resin during solid phase peptide synthesis or in solution via bio-orthogonal click chemistry to inverse electron demand Diels-Alder (IEDDA), Cu(I)-catalyzed azide-alkyne cycloaddition (CuAAc), or strain-promoted azide-alkyne cycloaddition (SPAAC) reactive covalent binding intermediates. Peptides can also efficiently be designed (in contrast to proteins) to either avoid self-reactivity with installed covalent binding groups by removing potentially reactive amino acids, or placing covalent binding groups at distal locations on the peptide to these potential self-reacting amino acids. The power of covalent binding chemistry in general is that it remains uniquely unreactive/kinetically stable, until positioned very close (within a few angstroms) to a partner reactive amino acid for a sufficiently long amount of time. This can only efficiently be achieved in dilute biological conditions and environments through non covalent reversible peptide-receptor binding interactions.

The inventors describe herein a series of covalent immune recruiters (CIRs) to allow dynamic targeting of FcR-expressing immune cells against the target defined by the target binding domain. The examples provided illustrate the use of this technology in a specific class of immune cell, CD64 expressing cells, and for two specific target proteins: PSMA, which can be used for targeting prostate cancer cells, and uPAR, which can be used for targeting multiple types of cancer cells. However, these tools can be adapted for targeting of other target proteins and for the treatment of other disease states in addition to cancer, including autoimmunity, allergy and transplant rejection.

I. Definitions

Unless otherwise indicated, the definitions and embodiments described in this and other sections are intended to be applicable to all embodiments and aspects of the present disclosure herein described for which they are suitable as would be understood by a person skilled in the art. Each aspect so defined may be combined with any other aspect or aspects unless clearly indicated to the contrary. In particular, any feature described herein may be combined with any other feature or features described herein.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either or both of those included limits are also included in the description.

As used herein and in the appended claims, the singular forms “a”, “an”, and “the” include plural references unless the context clearly dictates otherwise.

All numerical values within the detailed description and the claims herein are modified by “about” or “approximately” the indicated value, and take into account experimental error and variations that would be expected by a person having ordinary skill in the art.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In embodiments comprising an “additional” or “second” component, such as an additional or second compound, the second component as used herein is chemically different from the other components or first component. A “third” component is different from the other, first, and second components, and further enumerated or “additional” components are similarly different.

As used in this description and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

The term “suitable” as used herein means that the selection of the particular compound or conditions would depend on the specific manipulation to be performed, the identity of the molecule(s) to be transformed and/or the specific use for the compound, but the selection would be well within the skill of a person trained in the art.

The present description refers to a number of chemical terms and abbreviations used by those skilled in the art. Nevertheless, definitions of selected terms are provided for clarity and consistency.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “aryl” as used herein, whether it is used alone or as part of another group, refers to carbocyclic groups containing at least one aromatic ring and contains either 6 to 20 carbon atoms.

The term “azido” as used herein, whether it is used alone or as part of another group, refers to an azide, i.e. containing a monovalent —N₃ group.

The term “amine” or “amino,” as used herein, whether it is used alone or as part of another group, refers to groups of the general formula NR′R″, wherein R′ and R″ are each independently selected from hydrogen or C₁₋₆alkyl.

The term “amino acid” as used herein refers to an organic compound comprising amine (—NH₂) and carboxylic acid (—COON) functional groups, along with a side-chain specific to each amino acid. The common elements of an amino acid are carbon, hydrogen, oxygen and nitrogen, though other elements are found in the side-chains of certain amino acids, including S and Se. Unless otherwise specified, an amino acid referenced herein is one of the 23 proteinogenic amino acids, that is amino acids that are precursors to proteins, and are incorporated into proteins during translation.

The term “antibody” as used herein is intended to include chimeric and humanized antibodies and binding fragments thereof, including for example a single chain Fab fragment, Fab'2 fragment, or single chain Fv fragment. Humanized or other chimeric antibody fragments may include sequences from one or more than one isotype, class, or species. Further, these antibodies are typically produced as single domain antibody fragments, or as single chain antibodies in which the heavy and light chains are linked by a spacer. The antibodies may include sequences from any suitable species including human.

The term “antibody fragment” or “binding fragment” as used herein is intended to include without limitations Fab, Fab′, F(ab′)2, scFab, scFv, dsFv, ds-scFv, Fc-fusion proteins, dimers, minibodies, diabodies, and multimers thereof, multispecific antibody fragments and Domain Antibodies. Fab, Fab′ and F(ab′)2, scFv, dsFv, ds-scFv, Fc-fusion proteins, dimers, minibodies, diabodies, bispecific antibody fragments and other fragments can also be synthesized by recombinant techniques.

The term “functional variant” as used herein includes modifications of the compounds or polypeptide sequences disclosed herein that perform substantially the same function as the compound or polypeptide molecules disclosed herein in substantially the same way. For example, a functional variant polypeptide may comprise a sequence having at least 80%, or at least 90%, or at least 95% sequence identity to a sequence disclosed herein and which retains functional activity, for example binding to the cognate FcR or target protein. The functional variant polypeptide may also comprise conservatively substituted amino acid sequences of the sequences disclosed herein. Functional variants may also include non-peptide variants of, for example, a ligand or target binding domain described herein, for example a compound having a similar structure to the ligand or target binding domain and which retains functional activity, for example binding to the cognate FcR or target protein.

A “conservative amino acid substitution” as used herein, is one in which one amino acid residue is replaced with another amino acid residue without abolishing the protein's desired properties. Suitable conservative amino acid substitutions can be made by substituting amino acids with similar hydrophobicity, polarity, and R-chain length for one another. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue or non-natural amino acid in place of a non-derivatized residue provided that such polypeptide displays the requisite activity.

The term “sequence identity” as used herein refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=[number of identical overlapping positions]/[total number of positions]×100%). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The following symbol:

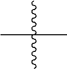

is used in chemical structures herein to represent a point of covalent attachment of a group to another group.

The term “aq.” as used herein refers to aqueous.

DMSO as used herein refers to dimethylsulfoxide.

DBCO as used herein refers to dibenzocyclooctyne.

SuFEx as used herein refers to sulfur(VI) fluoride exchange, or a functional group for sulfur(VI) fluoride exchange, including, without limitation, fluorosulfate, fluorosulfonate, or sulfonyl fluoride.

PSMA as used herein refers to prostate specific membrane antigen.

FTD as used herein refers to Fc targeting domain.

CBG as used herein refers to covalent binding group.

TBD as used herein refers to target cell binding domain or target binding domain.

CIR as used herein refers to covalent immune recruiter. The CIRs described herein are sometimes referred to as mCIRs or MCIRs.

(N)CIR or NCIR as used herein refers to non-covalent immune recruiter.

mCIR or MCIR as used herein refers to monocyte/macrophage covalent immune recruiter.

BLI as used herein refers to biolayer interferometry.

The term “linker” or “linker group” as used herein refers to any molecular structure that joins two or more other molecular structures together and that is compatible with a biological environment.

The term “compatible with a biological environment” as used herein it is meant that the chemical group or molecule is stable in, and/or does not denature or perturb, other molecules present in biological systems.

The term “biological systems” as used herein means any of a wide variety of systems which comprise proteins, enzymes, organic compounds, inorganic compounds, other sensitive biopolymers including DNA and RNA, and includes complex systems such as whole or fragments of plant, animal and microbial cells.

The term “subject” as used herein includes all members of the animal kingdom including mammals, and suitably refers to humans. Thus the methods and uses of the present disclosure are applicable to both human therapy and veterinary applications. Optionally, the term “subject” includes mammals that have been diagnosed with cancer or are in remission. In one embodiment, the term “subject” refers to a human having, or suspected of having, cancer.

The term “subject in need thereof” refers to a subject that could benefit from the method(s) or treatment(s) described herein, and optionally refers to a subject with cancer, or optionally a subject with increased risk of cancer, such as a subject previously having cancer, a subject with a precancerous syndrome or a subject with a strong genetic disposition.

The term “increased risk of cancer” as used herein means a subject that has a higher risk of developing a particular cancer than the average risk of the population. A subject may have a higher risk due to previously having had said particular cancer and/or having a genetic risk factor for said particular cancer or exhibit a pre-cancer syndrome.

The term “administered” or “administering” as used herein means administration of a therapeutically effective amount of one or more CIRs or functionalized cells described herein, or a composition described herein to a patient.

The term “immune response” as used herein can refer to activation of either or both the adaptive and innate immune system cells such that they shift from a dormant resting state to a state in which they are able to elaborate molecules typical of an active immune response.

The phrase “inducing an immune response” as used herein refers to a method whereby an immune response is activated. The phrase “enhancing an immune response” refers to augmenting an existing immune response.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di-and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds for laboratory use, or for subsequent conversion to a pharmaceutically acceptable acid addition salt.

A base addition salt suitable for, or compatible with, the treatment of subjects is any non-toxic organic or inorganic base addition salt of any acidic compound. A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

The term “solvate” as used herein means a compound, or a salt of a compound, wherein molecules of a suitable solvent are incorporated in the crystal lattice. Solvates of CIRs described herein include, for example, those made with solvents that are pharmaceutically acceptable. Examples of such solvents include water (resulting solvate is called a hydrate) and ethanol and the like. Suitable solvents are physiologically tolerable at the dosage administered.

The term “prodrug” as used herein means a compound, or salt and/or solvate of a compound, that, after administration, is converted into an active drug.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. For example, a subject with early cancer can be treated to prevent progression, or alternatively a subject in remission can be treated with one or more CIRs or functionalized cells described herein to prevent recurrence. Treatment methods comprise administering to a subject a therapeutically effective amount of one or more CIRs or functionalized cells described herein and optionally consist of a single administration, or alternatively comprise a series of administrations.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a subject becoming afflicted with a disease, disorder or condition or manifesting a symptom associated with a disease, disorder or condition.

The term “disease, disorder or condition” as used herein refers to a disease, disorder or condition treatable by immunotherapy, such as by one or more CIRs or functionalized cells described herein.

The term “immunotherapy” as used herein refers to the treatment of disease, disorder or condition by activating the immune system to produce or provoke an immune response.

As used herein, the term “effective amount” or “therapeutically effective amount” means an amount of one or more CIRs or functionalized cells described herein that is effective, at dosages and for periods of time necessary to achieve the desired result. For example, in the context of treating a disease, disorder or condition mediated or treatable by immunotherapy, an effective amount is an amount that, for example, provoke an immune response compared to without administration of the one or more CIRs or functionalized cells described herein.

As used herein, the term “cancer” refers to one of a group of diseases caused by the uncontrolled, abnormal growth of cells that can spread to adjoining tissues or other parts of the body. Cancer cells can form a solid tumor, in which the cancer cells are massed together, or exist as dispersed cells, as in a hematological cancer such as leukemia.

The term “cancer cell” refers a cell characterized by uncontrolled, abnormal growth and the ability to invade another tissue or a cell derived from such a cell. Cancer cells include, for example, a primary cancer cell obtained from a patient with cancer or cell line derived from such a cell.

The phrase “cancer burden” refers to the quantum of cancer cells or cancer volume in a subject. Reducing cancer burden accordingly refers to reducing the number of cancer cells or the cancer volume in a subject.

II. Covalent Immune Recruiters, Precursor Compounds, Cells, and Compositions

Described herein is the development of covalent immune recruiters useful for engineering immune cells, such as FcR-expressing immune cells, to be functionalized with a target binding domain. Also provided herein are precursor compounds for generating a CIR described herein. Further provided herein are functionalized cells modified using a CIR described herein.

As shown herein, both non-covalent and covalent peptides bind to CD64 with high selectivity and reasonably fast kinetics. The ability of CIR to form a ternary complex in vitro, and to induce phagocytosis in flow cytometry is also demonstrated herein. CD64 is a key macrophage/monocyte activation FcR responsible for anti-tumor ADCP. The model peptide “CP33” is a peptide known to bind and activate CD64 dependent ADCP of prostate cancer cells when incorporated into a bi-functional synthetic immunotherapeutic asset known as “Synthetic Antibody Mimics” or “SyAMs”. As described herein, the CP33 peptide has been engineered to covalently bind human CD64 using SuFEx covalent binding groups, which will enable substantial enhancements in anti-tumor ADCP function with a therapeutic that is a fraction of the molecular weight of therapeutic antibodies and SyAMs.

### Covalent Immune Recruiters

The covalent immune recruiters described herein comprise an Fc receptor (FcR) targeting domain (FTD), which can bind a cognate FcR, a covalent binding group (CBG) which comprises a functional group that, on binding of the FTD to the cognate FcR, forms a covalent bond with an amino acid in the FcR, and a target binding domain (TBD) which binds a target protein. As shown in the Examples herein, a CIR comprising an FTD such as CP33, or functional variants thereof, and a target binding domain such as glutamate urea lysine (GUL) can be used to functionalize an FcR, such as CD64, to bind a target protein such as PSMA. As further shown in the Examples herein, a CIR comprising an FTD such as CP33, or functional variants thereof, and a target binding domain such as a synthetic uPAR binding peptide ligand can be used to functionalize an FcR, such as CD64, to bind a target protein such as uPAR. Similarly, as shown in the Examples herein, a CIR comprising an FTD such as CP33 or variants thereof, and a TBD such as biotin, functionalizes a monocyte to target streptavidin beads for phagocytosis. In some embodiments, the CIR comprises more than one TBD, optionally two TBDs, three TBDs, or more than three TBDs.

### FcR Targeting Domains

The FcR targeting domain (FTD) is a chemically synthetic molecule comprising a binding domain which binds to a cognate FcR, on an immune cell. Any suitable FTD may be used depending on the specific FcR being targeted. In an embodiment, the FTD binds FcγR, optionally selected from CD64, CD32, CD16a, and CD16b.

In an embodiment, the FcR is CD64. In an embodiment, the FTD comprises circular peptide 33 (CP33) having the sequence VNSCLLLPNLLGCGDD (SEQ ID NO: 1), wherein C4 and C13 form a disulfide bond, or a functional variant thereof. In an embodiment, the FTD comprises a circular peptide 33 (CP33) having the sequence VNSCLLLPNLLGCDGD (SEQ ID NO: 2), wherein C4 and C13 form a disulfide bond, or a functional variant thereof. In an embodiment, the FTD comprises a CP33 having the sequence of SEQ ID NO: 3, SEQ ID NO: 4, SEQ ID NO: 7, SEQ ID NO: 8, SEQ ID NO: 9, SEQ ID NO: 10, SEQ ID NO: 11, or SEQ ID NO: 12, or functional variants thereof. In an embodiment, CP33 comprises a peptide having the sequence of SEQ ID NO: 3, wherein X is K and wherein C5 and C14 form a disulfide bond. In an embodiment, CP33 comprises a peptide having the sequence of SEQ ID NO: 4, wherein X is K and wherein C5 and C14 form a disulfide bond. In an embodiment, CP33 comprises a peptide having the sequence of SEQ ID NO: 7, wherein X is K and wherein C7 and C16 form a disulfide bond. In an embodiment, CP33 comprises a peptide having the sequence of SEQ ID NO: 8, wherein X is K and wherein C7 and C16 form a disulfide bond. In an embodiment, CP33 comprises a peptide having the sequence of SEQ ID NO: 9, wherein X is K and wherein C5 and C14 form a disulfide bond. In an embodiment, CP33 comprises a peptide having the sequence of SEQ ID NO: 10, wherein X is K and wherein C5 and C14 form a disulfide bond. In an embodiment, CP33 comprises a peptide having the sequence of SEQ ID NO: 11, wherein X1 is K, wherein X18 is K, and wherein C5 and C14 form a disulfide bond. In an embodiment, CP33 comprises a peptide having the sequence of SEQ ID NO: 12, wherein X1 is K, wherein X18 is K, and wherein C5 and C14 form a disulfide bond. In an embodiment, the CP33 peptide is C-terminally amidated. In an embodiment, the CP33 peptide is not C-terminally amidated.

### Target Cell Binding Domains (TBD)

Target cell binding domains, or target binding domains (TBDs), comprise targeting moieties which are taken up and retained in a particular site of a subject such as a biological structure for example an organ or tissue or a pathological structure for example a tumor. Optionally, there is little or no accumulation and/or retention in non-target sites over a particular time period. In an embodiment, the TBD binds to a protein, for example a protein that is overexpressed in a disease, disorder or condition such as cancer. For example, the TBD may bind prostate specific membrane antigen (PSMA), FRa, CD38 and/or uPAR expressed in a cancer such as in breast cancer, ovarian cancer, esophageal cancer, multiple myeloma, and/or GBM. Targeting moieties are known and the selection of a suitable targeting moiety for a particular therapeutic use can be made by a person skilled in the art. Targeting moieties include, but are not limited to, small molecules such as protein binding compounds, peptides, enzyme inhibitors, receptor ligands, or pharmaceutical-like compounds.

In an embodiment, the TBD comprises a moiety that binds to antigens on the surface of a target cell. In an embodiment, the TBD is a glutamate urea lysine (GUL) ligand, or a functional variant thereof, that binds to prostate specific membrane antigen (PSMA).

In an embodiment, the TBD comprises any of the functional variants of a GUL ligand (“cell binding moiety”) described in U.S. Pat. No. 9,296,708. Accordingly, the TBD group may comprise one of the following groups:

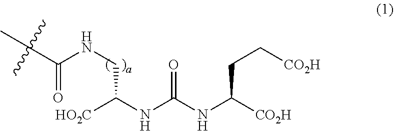

wherein a is an integer from 0 to 10, 1 to 15, 1 to 10, 1 to 8, or 1, 2, 3, 4, 5 or 6;

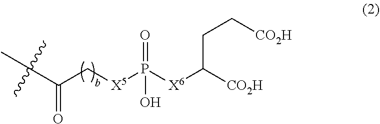

wherein X 5 and X 6 are independently CH₂, O, NH or S; and
b is an integer from 0 to 10, 1 to 15, 1 to 10, 1 to 8, or 1, 2, 3, 4, 5 or 6;

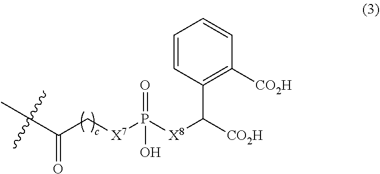

wherein X 7 and X 8 are independently CH₂, O, NH or S; and c is an integer from 0 to 10, 1 to 15, 1 to 10, 1 to 8, or 1, 2, 3, 4, 5 or 6; or

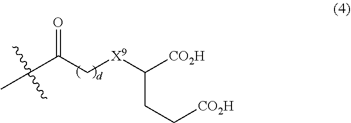

wherein X⁹ is O, CH₂, NR⁵, S(O), SO₂, SO₂O, OSO₂ or OSO₂O;
R⁵ is H, C₁₋₄alkyl or C(O)C₁₋₄alkyl; and
d is an integer from 0 to 10, 1 to 15, 1 to 10, 1 to 8, or 1, 2, 3, 4, 5 or 6.

In an embodiment, a, b, c, and d are independently 1, 2, 3, 4, 5 or 6, suitably 2, 3 or 4, more suitably 4.

In an embodiment, the TBD comprises:

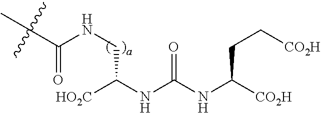

wherein a is 1, 2, 3, 4, 5 or 6.

In an embodiment, the TBD comprises biotin, or a functional variant thereof. In an embodiment, the functional variant of biotin comprises the following group:

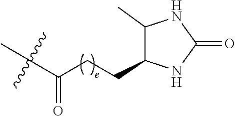

wherein e is 1, 2, 3, 4, 5 or 6.

In some embodiments, the TBD comprises other tumor antigen binding ligands such as, without limitation, synthetic peptides which bind uPAR (e.g. as described in Ploug M, et al., Peptide-derived antagonists of the urokinase receptor, affinity maturation by combinatorial chemistry, identification of functional epitopes, and inhibitory effect on cancer cell intravasation. Biochemistry. 2001 Oct. 9;40(40):12157-68, incorporated herein by reference, or functional variants thereof). In an embodiment, the synthetic peptide which binds uPAR comprises an amino acid sequence of L-Lys-Gly-Gly-L-Ser-Gly-L-Asp-L-Cha-L-Phe-D-Ser-D-Arg-L-Tyr-L-Leu-L-Trp-L-Ser (SEQ ID NO: 15), or a functional variant thereof. In an embodiment, the synthetic peptide which binds uPAR comprises an amino acid sequence of L-Lys-Gly-Gly-L-Ser-Gly-L-Asp-L-Cha-L-Phe-D-Ser-D-Arg-L-Ala-L-Leu-L-Trp-L-Ser (SEQ ID NO: 16) as used herein, or functional variant thereof. In some embodiments, the TBD comprises other tumor antigen binding ligands such as, without limitation, HER2, folate receptor binding molecules such as folate or methotrexate, Toll-like receptor (TLR) agonists, or PD-1/PD-L1 antagonists. The TBD may also include Integrin binding ligands such as the Knottin peptide and other RDG mimetics. The TBD may also include therapeutic antibodies, scFv, aptamers, and other biologics.

In some embodiments, the TBD may be an antigen or epitope that binds to allergen-reactive B cells such as, without limitation, Ara h1, Ara h2, Fel d1 or other defined allergens, antigens and epitopes for allergen-reactive B cells.

In some embodiments, the TBD may be an antigen or epitope that binds autoreactive B cells such, without limitation, citrullinated peptides, desmogleins or other antigens and epitopes for autoreactive B cells.

In some embodiments, the TBD may be a ligand for a donor MHC molecule that would be used to elicit a regulatory T cell response to suppress graft rejection.

In some embodiments, the CIR may comprise more than one TBD, optionally two TBDs, three TBDs, or more than three TBDs.

### Covalent Binding Groups

The covalent binding group (CBG) of the CIRs described herein comprises a functional group that reacts with and forms a covalent bond with an amino acid in the FcR being targeted. Depending on the CBG and type of reaction, the formation of the covalent bond may result in elimination of the FcR targeting domain.

The covalent binding group comprises a functional group that reacts with an amino acid in the FcR (e.g. CD64) to form a covalent bond with the FcR. In an embodiment, the reaction results in elimination (or displacement) of the FcR targeting domain. In an embodiment, the reaction does not result in elimination (or displacement) of the FcR targeting domain. The amino acid in the FcR forming a covalent bond with the CIR is proximal to the binding site for the FcR targeting domain. By “proximal” it is meant that the amino acid is located in an area that, when the CIR is bound to the FcR via the FcR targeting domain, the amino acid is in a spatial location to react with the covalent binding group. For example, the distance between the amino acid and the covalent binding group may be about 2 Å to about 10 Å.

A wide range of suitable functional groups can be used to provide covalent attachment, including, for example, acylimidazole esters, sulfonyl fluoride exchange chemistries, in addition to a number of known proximity activated acylation and alkylation chemistries typically employed in the development of covalent drugs and enzyme inhibitors such as alpha haloketones, vinyl sulfones and sulfonamides, acrylamides, tosyl sulfones, boronic acids/esters, and carbonyl compounds.

In some embodiments, the covalent binding group comprises a sulfur(VI) fluoride exchange (SuFEx) group, for example fluorosulfate, fluorosulfonate, or sulfonyl fluoride.

In some embodiments, the covalent binding group comprises an electrophilic functional group that reacts with an amino acid nucleophile in a nucleophilic substitution reaction. In an embodiment, the amino acid nucleophile is an amine (NH₂) or a thiol (SH). In an embodiment, the electrophilic functional group in the covalent binding group comprises an imidazole group having the following structure:

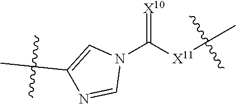

wherein
X¹⁰ is S, O or NR⁶;
X¹¹ is O or NR⁷; and
R⁶ and R⁷ are independently H or C₁₋₄alkyl. In an embodiment, X¹⁰ and X¹¹ are both O.

The covalent binding group may also incorporate click chemistry handles to enable for a subsequent 2-step ligation of the target binding domain to the acceptor on the FcR using “click” chemistry processes. Click chemistry is used in the art to describe a class of “bio-orthogonal” reactions which are often used for attaching a probe or substrate of interest to a specific biomolecule in a process called bioconjugation which can take place in vitro or directly in vivo. This class of biocompatible small molecule reactions may include, for example, [3+2] cycloadditions such as the Huisgen 1,3-dipolar cycloaddition, thiol-ene reactions, Diels-Alder reactions and inverse electron demand Diels-Alder reactions, [4+1] cycloadditions between isonitriles (isocyanides) and tetrazines. In some embodiments, the click chemistry is a copper catalyzed reaction of an azide and an alkyne to form a triazole.

Other functional groups may be used in the covalent binding group. Such group would be compatible with a biological environment and would react with a functional group on an amino acid in an protein to form a covalent bond, optionally wherein formation of the covalent bond results in elimination of the receptor targeting domain.

### Linker Groups

The CIRs described herein may also comprise one or more linkers. A linker may be incorporated to provide appropriate distance and/or spatial orientation of the covalent binding group and the FcR targeting domain to optimize covalent binding to the FcR. A linker may also be incorporated to provide appropriate distance and/or spatial orientation of the target binding domain and the covalent binding group. In some embodiments, the linker rigidity and length is tuned to maximize labeling kinetics and further comprises rigidifying elements such as carbocycles, heterocycles, aromatics and/or heteroaromatics.

### Precursor Compounds

Also provided herein are precursor compounds for generating a CIR described herein. For example, precursor compounds include a) an FcR targeting component comprising an FcR targeting domain, a covalent binding group, and an acceptor group for covalent attachment of the TBD, and b) a target binding component comprising a target binding domain and a cognate donor group for covalent attachment to the acceptor group of the FcR targeting component.

The acceptor group and donor group will interact and react under suitable conditions to result in the formation of a covalent bond. Any suitable acceptor group and donor group may be used, for example an azido group can be used with a DBCO group. Other sets of “click chemistry” groups can be used, for example, without limitation, tetrazine may be used with transcyclooctene.

### Functionalized Cells

The CIR is designed to be used to functionalize cells that express the cognate FcR. As used herein, “functionalized cells” refers to immune cells that have been modified by one or more CIRs described herein. Accordingly, functionalized cells comprise an FcR covalently linked to a TBD. In an embodiment, the functionalized cells comprise an FcR covalently linked to more than one TBDs.

Any suitable immune cell expressing an FcR may be used. In an embodiment, the FcR is a native FcR (e.g. an FcR that is endogenously expressed on the immune cell). Functionalized cells of a specified type may be described as being derived from the specified cell type, for example functionalized immune cells may be described as being derived from immune cells.

In an embodiment, the immune cells can suppress tissue pathology (e.g. suppress tumor growth, suppress graft rejection, suppress allergic reactions, suppress autoimmune reactions). In an embodiment, the immune cells are lymphocytes, monocytes, polymorphonuclear cells, erythrocytes or megakaryocytes. In an embodiment, the immune cells are B cells, NK cells, macrophages, neutrophils, basophils, or eosinophils.

In an embodiment, the FcR is an FcγR. In an embodiment, the FcγR is CD64.

In an embodiment, the cells are taken from a human, functionalized ex vivo, and delivered to an unrelated human. In such embodiments, the cells are allogenic. As used herein, the term “allogenic” refers to cells, tissue, DNA, or factors originally obtained (taken or derived) from a subject who is a different individual than the intended recipient of said cells, but who is of the same species as the recipient. Optionally, allogenic cells may be cells from a cell culture. For example, in the context where allogenic functionalized cells are administered to a subject, cells removed from an individual of the same species that is not the subject are functionalized with a desired CIR, and the functionalized cells are administered to the subject. In one embodiment, the cells are allogenic cells obtained from a healthy donor.

In an embodiment, the cells are taken from a human, functionalized ex vivo, and given back to the same human. In such embodiments, the cells are autologous. As used herein, the term “autologous” refers to cells, tissue, DNA or factors originally obtained taken or derived from an individual's own tissues, cells or DNA. For example, in the context where autologous functionalized cells are administered to a subject, cells removed from the subject are functionalized with a desired CIR, and the functionalized cells are administered to the subject.

In alternative embodiments, the immune cells may be functionalized directly within the individual to be treated using CIRs formulated for in vivo delivery.

### Compositions

The CIR or functionalized cells described herein are suitably formulated in a conventional manner into compositions using one or more carriers or diluents. Accordingly, the present description also includes a composition comprising one or more CIRs or functionalized cells described herein and a carrier or diluent. The CIRs or functionalized cells described herein are suitably formulated into pharmaceutical compositions for administration to subjects in a biologically compatible form suitable for administration in vivo. Accordingly, the present description further includes a pharmaceutical composition comprising the CIRs or functionalized cells described herein, and a pharmaceutically acceptable carrier. In some embodiments the pharmaceutical compositions are used in the treatment of any of the diseases, disorders or conditions described herein.

The CIRs or functionalized cells described herein may be administered to a subject in a variety of forms depending on the selected route of administration, as will be understood by those skilled in the art. For example, the CIRs or functionalized cells described herein may be administered by oral, inhalation, parenteral, buccal, sublingual, nasal, rectal, vaginal, patch, pump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20^{th} edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, the CIRs or functionalized cells described herein are orally administered, for example, with an inert diluent or with an assimilable edible carrier, or it is enclosed in hard or soft shell gelatin capsules, or it is compressed into tablets, or it is incorporated directly with the food of the diet. In some embodiments, the CIR or functionalized cells described herein is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

In some embodiments, the CIRs or functionalized cells described herein are administered parenterally. For example, solutions of one or more CIRs or functionalized cells described herein are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the CIRs or functionalized cells described herein are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, the CIRs or functionalized cells described herein are formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, CIRs or functionalized cells described herein are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the CIRs or functionalized cells described herein are conveniently delivered in the form of a solution, dry powder formulation or suspension from a pump spray container that is squeezed or pumped by the subject or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound and a suitable powder base such as lactose or starch. The aerosol dosage forms can also take the form of a pump-atomizer.

Compositions suitable for buccal or sublingual administration include tablets, lozenges, and pastilles, wherein a compound is formulated with a carrier such as sugar, acacia, tragacanth, or gelatin and glycerine. Compositions for rectal administration are conveniently in the form of suppositories containing a conventional suppository base such as cocoa butter.

Suppository forms of the CIRs or functionalized cells described herein are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16^{th} Ed., Mack Publishing, Easton, PA, 1980, pp. 1530-1533 for further discussion of suppository dosage forms.

In some embodiments the CIRs or functionalized cells described herein are coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The CIRs or functionalized cells described herein, including pharmaceutically acceptable salts and/or solvates thereof, are suitably used on their own but will generally be administered in the form of a pharmaceutical composition in which the one or more CIRs or functionalized cells described herein (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

### Kits

Also provided herein are kits comprising one or more of the CIRs or functionalized cells described herein along with a suitable container or packaging and/or instructions for use thereof, such as, for example, for use in the methods or applications described herein. For example the instructions may relate to generating a functionalized cell, and/or treating a disease, disorder or condition treatable by immunotherapy. In an embodiment, the kit further comprises an applicator or other suitable delivery device. In an embodiment, the kit further comprises one or more additional drugs.

Also provided herein are kits for generating a CIR, the kit comprising one or more precursor compounds for generating a CIR along with a suitable container or packaging therefor, and/or instructions for use thereof. In an embodiment, the kit comprises an FcR targeting component comprising an FcR targeting domain, a covalent binding group, and an acceptor group for covalent attachment of the TBD. In an embodiment, the kit comprises a target binding component comprising a target binding domain and a cognate donor group for covalent attachment to the acceptor group of the FcR targeting component. In an embodiment, the kit comprises an FcR targeting component and a target binding component.

III. Methods and Uses

Also described herein are methods for generating the CIRs described herein, comprising contacting an FcR targeting component comprising an FcR targeting domain, a covalent binding group, and an acceptor group for covalent attachment of the TBD, with a target binding component comprising a TBD and a cognate donor group for covalent interaction with the acceptor group of the FcR targeting component, wherein the FcR targeting component and the target binding component are contacted under suitable conditions to allow binding and covalent attachment of the acceptor group and cognate donor group, thereby generating a CIR.

As shown herein, functionalized cells comprising a target binding domain covalently linked to a FcR can be generated by contacting immune cells expressing an FcR with a suitable covalent immune recruiter (CIR) described herein comprising a cognate FcR targeting domain, a covalent binding group, and the target binding domain (TBD). As will be understood, the suitability of a given TBD will depend on the intended application or use of the functionalized cell. For example, where the intended application of the functionalized cell is for the treatment of a specific cancer, a suitable TBD will be one that binds cells of the specific cancer. The skilled person can readily select a suitable TBD for the intended application or use.

Accordingly, an aspect described herein includes a method for generating a functionalized cell, the method comprising providing a cell expressing an FcR, and contacting the cell with a suitable cognate CIR (comprising a cognate FcR targeting domain that interacts with the FcR) under suitable conditions to allow binding of the FcR targeting domain and FcR, and covalent attachment of the covalent binding group to the FcR, thereby generating a functionalized cell.

Functionalized cells may be generated for example in vitro, ex vivo, or in vivo. For example, an immune cell may be contacted in vitro or ex vivo with a suitable cognate CIR. Alternatively, the CIR may be administered to a subject, and the functionalized cell generated in vivo.

The functionalized cells described herein may be directed towards a target cell and exhibit a cytotoxic response to, or phagocytosis of, the target cell.

Accordingly, the functionalized cells described herein are effective tools for directing the functionalized cell to a target cell, and/or triggering a cytotoxic response to, or phagocytosis of, the target cell. The target cell may be any desired cell. For example, in the context of cancer therapy, the target cell may be a cancer cell.

Also described herein is a method for recruiting a functionalized cell to a target cell in a subject, comprising administering an effective amount of a) a CIR; or b) a functionalized cell to the subject.

Further described herein is a method for targeting and/or recruiting a functionalized cell for provoking an immune response to a target cell in a subject, comprising administering an effective amount of a) a CIR comprising a suitable TBD; or b) a functionalized cell comprising a suitable TBD to the subject. An aspect also includes use of a) a CIR comprising a suitable TBD; or b) a functionalized cell comprising a suitable TBD for provoking an immune response to a target cell in a subject. An aspect also includes use of a) a CIR comprising a suitable TBD; or b) a functionalized cell comprising a suitable TBD in the manufacture of a medicament for provoking an immune response to a target cell in a subject. An aspect also includes a) a CIR comprising a suitable TBD; or b) a functionalized cell comprising a suitable TBD for use in provoking an immune response to a target cell in a subject.

Also described herein is a method for provoking cellular phagocytosis of a target cell in a subject, comprising administering an effective amount of a) a CIR comprising a suitable TBD; or b) a functionalized cell comprising a suitable TBD to the subject. An aspect also includes use of a) a CIR comprising a suitable TBD; or b) a functionalized cell comprising a suitable TBD for provoking cellular phagocytosis of a target cell in a subject. An aspect also includes use of a) a CIR comprising a suitable TBD; or b) a functionalized cell comprising a suitable TBD in the manufacture of a medicament for provoking cellular phagocytosis of a target cell in a subject. An aspect also includes a) a CIR comprising a suitable TBD; or b) a functionalized cell comprising a suitable TBD for use in provoking cellular phagocytosis of a target cell in a subject.

Also described herein are methods of: recruiting an immune cell for immunotherapy, recruiting an immune cell for provoking an immune response to a target cell, targeting an immune cell for provoking an immune response to a target cell, and provoking cellular phagocytosis of a target cell.

Further described herein is a use of the CIR or functionalized cells described herein for the preparation of a medicament for the methods and uses described herein, for example the treatment of a disease, disorder, or condition. Such diseases, disorders, or conditions that are treatable by immunotherapy include, without limitation, cancer, autoimmune diseases, and allergy, and transplant rejection.

Also described herein is a method of treating a disease, disorder or condition that is treatable by provoking an immune response, comprising administering a therapeutically effective amount of a) a CIR comprising a suitable TBD; or b) a functionalized cell comprising a suitable TBD to a subject in need thereof. Such diseases, disorders, or conditions that are treatable by immunotherapy include, without limitation, cancer, autoimmune diseases, and allergy, and transplant rejection.

Also described herein is a use of a) a CIR comprising a suitable TBD; or b) a functionalized cell comprising a suitable TBD for treating or preventing a disease, disorder or condition treatable by immunotherapy. An aspect also includes use of a) a CIR comprising a suitable TBD; or b) a functionalized cell comprising a suitable TBD for the preparation of a medicament for treating or preventing a disease, disorder or condition treatable by immunotherapy. The disclosure further includes a) a CIR comprising a suitable TBD; or b) a functionalized cell comprising a suitable TBD, for use in treating or preventing a disease, disorder or condition treatable by immunotherapy. Such diseases, disorders, or conditions that are treatable by immunotherapy include, without limitation, cancer, autoimmune diseases, and allergy, and transplant rejection.

In an embodiment, the disease, disorder or condition treatable by immunotherapy is cancer, therefore the present disclosure includes a method of treating cancer comprising administering a therapeutically effective amount of a) a CIR comprising a target binding domain (TBD) that binds the cancer being treated; or b) a functionalized cell comprising a TBD that binds the cancer being treated to a subject in need thereof. The present disclosure also includes a use of a) a CIR comprising a suitable TBD; or b) a functionalized cell comprising a suitable TBD for treatment of cancer as well as a use of a) CIR comprising a suitable TBD; or b) a functionalized cell comprising a suitable TBD for the preparation of a medicament for treatment of cancer. The disclosure further includes a) a CIR comprising a suitable TBD; or b) a functionalized cell comprising a suitable TBD for use in treating cancer. In an embodiment, the CIR or functionalized cell comprising a suitable TBD is administered for the prevention of cancer in a subject such as a mammal having a predisposition for cancer.

In an embodiment, the cancer is one that is impacted or treatable by immunotherapy. In an embodiment, the cancer is one that is impacted or treatable by activation of immune cells. In an embodiment, the cancer is one that is impacted or treatable by provoking an immune response to tumor cells. In an embodiment, the cancer is one that is impacted or treatable by provoking phagocytosis of tumor cells.

In an embodiment, the cancer is selected from, but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

In an embodiment, the cancer is selected from prostate cancer, breast cancer, ovarian cancer and glioblastoma. In an embodiment, the cancer is prostate cancer.

In further embodiments, the present disclosure also includes a method of treating a disease, disorder or condition treatable by immunotherapy, comprising administering to a subject in need thereof a therapeutically effective amount of a) a CIR comprising a suitable TBD; or b) a functionalized cell comprising a suitable TBD, in combination with another agent useful for treatment of the disease, disorder or condition treatable by immunotherapy. The present disclosure also includes a use of a) a CIR comprising a suitable TBD; or b) a functionalized cell comprising a suitable TBD, in combination with an agent useful for treatment of a disease, disorder or condition treatable by immunotherapy, for treatment of such disease, disorder or condition.

In a further embodiment, the disease, disorder or condition treatable by immunotherapy is cancer and the a) CIR comprising a suitable TBD; or b) functionalized cell comprising a suitable TBD, are administered in combination with one or more additional cancer treatments. In another embodiment, the additional cancer treatment is selected from radiotherapy, chemotherapy, targeted therapies such as antibody therapies and small molecule therapies such as tyrosine-kinase and serine-threonine kinase inhibitors, immunotherapy, hormonal therapy and anti-angiogenic therapies.

Effective amounts vary according to factors such as the disease state, age, sex and/or weight of the subject. Accordingly, the amount of a given CIR or functionalized cell described herein that will correspond to an effective amount will vary depending upon factors, such as the given CIR or functionalized cell described herein, the pharmaceutical formulation, the route of administration, the type of condition, disease or disorder, the identity of the subject being treated, and the like, but can nevertheless be routinely determined by one skilled in the art.

Suitable administration schedules may include, without limitation, at least once a week, from about one time per two weeks, three weeks or one month, about one time per week to about once daily, 2, 3, 4, 5 or 6 times daily. The length of the treatment period may depend on a variety of factors, such as the severity of the disease, disorder or condition, the age of the subject, the concentration and/or the activity of the CIR or functionalized cells described herein and/or a combination thereof. It will also be appreciated that the effective dosage of the CIR or functionalized cells described herein used for the treatment may increase or decrease over the course of a particular treatment regime. Changes in dosage may result and become apparent by standard diagnostic assays known in the art. In some instances, chronic administration is required. For example, the CIR or functionalized cells described herein are administered to the subject in an amount and for duration sufficient to treat the subject.

In an embodiment, the subject is a mammal. In another embodiment, the subject is human.

The CIR or functionalized cells described herein may be either used alone or in combination with other known agents useful for treating diseases, disorders or conditions as defined above. When used in combination with other agents useful in treating such diseases, disorders or conditions, the CIR or functionalized cells described herein may be administered contemporaneously with those agents. As used herein, “contemporaneous administration” of two substances to a subject means providing each of the two substances so that they are both active in the individual at the same time. The exact details of the administration will depend on the Pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. In other embodiments, the combination of agents is administered to a subject in a non-contemporaneous fashion. In an embodiment, the CIR, functionalized cell, composition, etc. described herein is administered with another therapeutic agent simultaneously or sequentially in separate unit dosage forms or together in a single unit dosage form. Accordingly, the present description provides a single unit dosage form comprising the CIR, functionalized cell, composition, etc. described herein, an additional therapeutic agent, and a pharmaceutically acceptable carrier.

The dosage of the CIR or functionalized cells described herein varies depending on many factors such as the pharmacodynamic properties of the compound/cell, the mode of administration, the age, health and weight of the recipient, the nature and extent of the symptoms, the frequency of the treatment and the type of concurrent treatment, if any, and the clearance rate of the compound/cell in the subject to be treated. One of skill in the art can determine the appropriate dosage based on the above factors. In some embodiments, CIR or functionalized cells described herein is administered initially in a suitable dosage that is adjusted as required, depending on the clinical response. Dosages will generally be selected to maintain sufficient levels of the CIR, functionalized cell, composition, etc. described herein.

### EXAMPLES

The following non-limiting examples are illustrative of the present disclosure.

### Example 1: Design of CP33 Mutants

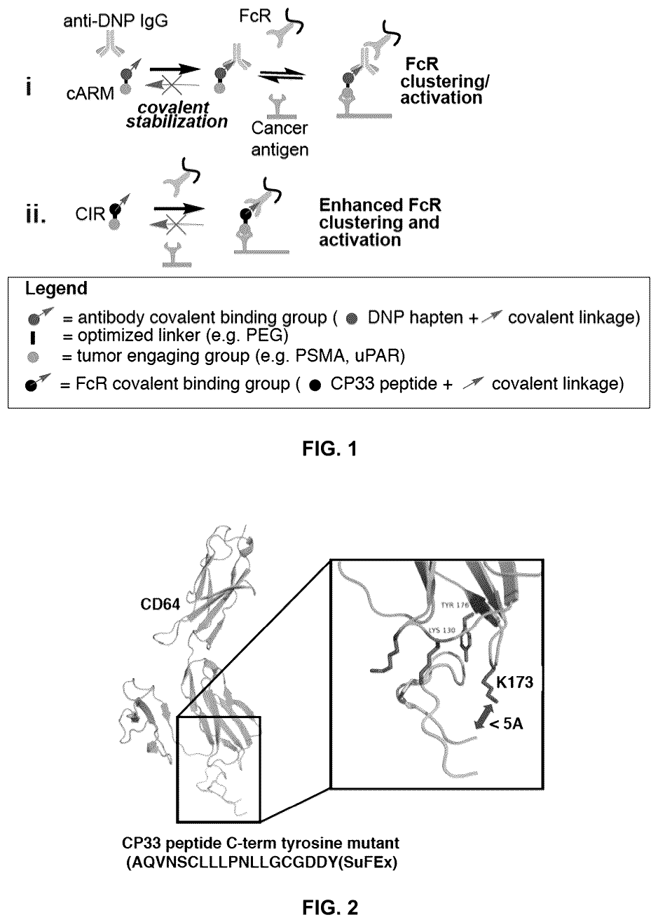

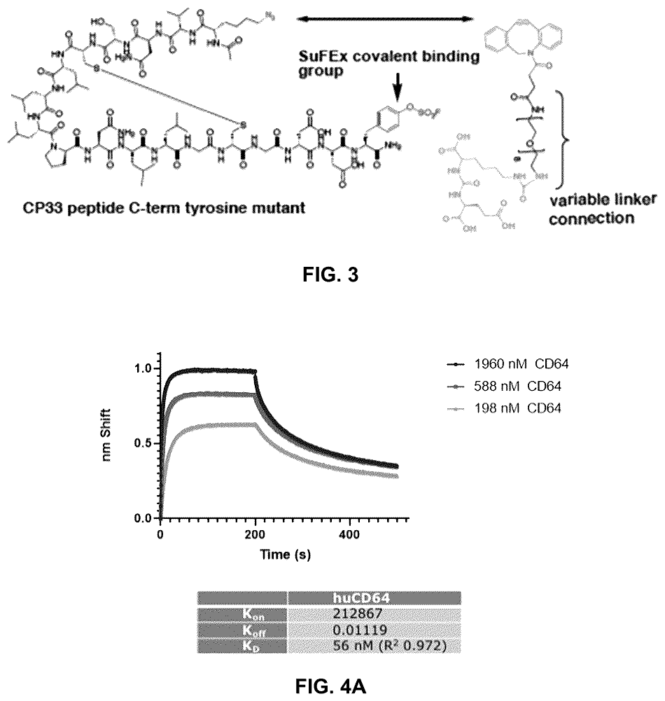

The optimal location to install SuFEx covalent chemistry was informed by computation guided design of CD64 receptor binding to CP33 peptide (FIG. 2). The goal was to incorporate SuFEx at a position on CP33 peptide that is pre-organized for covalent reaction with proximal nucleophilic amino acids on CD64. By docking the CP33 peptide onto a known crystal structure of CD64, one optimal location on CP33 was identified at the N terminus to incorporate SuFEx covalent binding groups via insertion of a tyrosine mutation into the CP33 sequence. This enables for orthogonal incorporation of SuFEx at the tyrosine hydroxyl directly on solid support SPPS resin (FIG. 3). The N terminus of CP33 was selected due to the resultant very close proximity of the installed SuFEx to potentially reactive lysine and tyrosine amino acids on CD64. Notably, SuFEx represents one highly strategic covalent binding group due to the following: a) it can be easily incorporated into CIRs in organic solvents without side reactions due to unique reactivity that dramatically increases in aqueous solvent; b) it is kinetically stable and can undergo dramatic proximity induced rate enhancements; c) it is stable to hydrolysis in vivo; d) it can react with several amino acids and not just cysteine/lysine akin to many other covalent binding groups; and e) several SuFEx moieties exist including fluorosulfonyl and aryl sulfonofluorate which have diverse range of reactivity and selectivity, allowing tunability of CIRs.

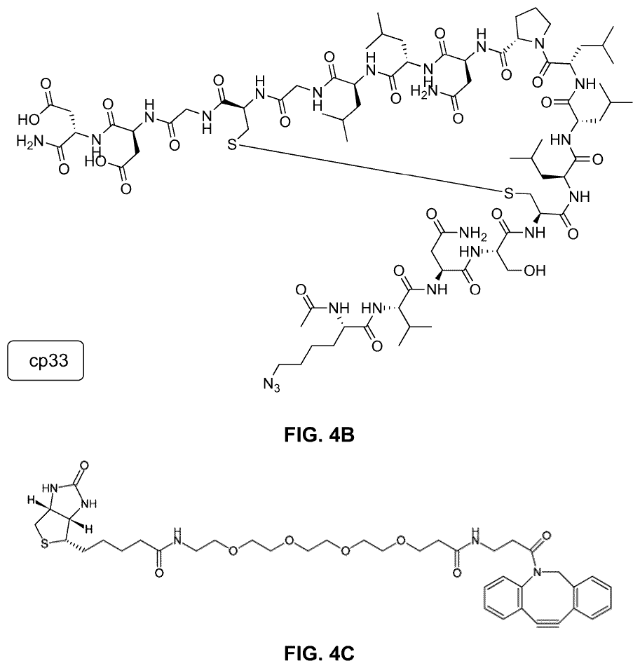

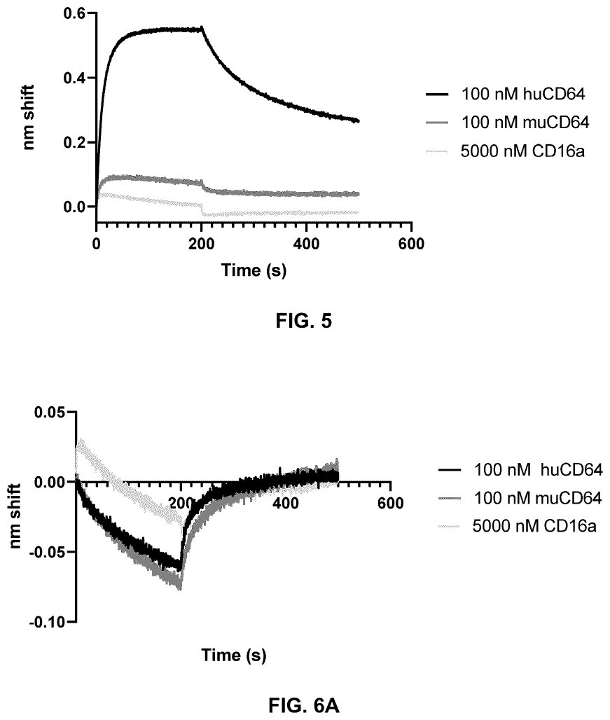

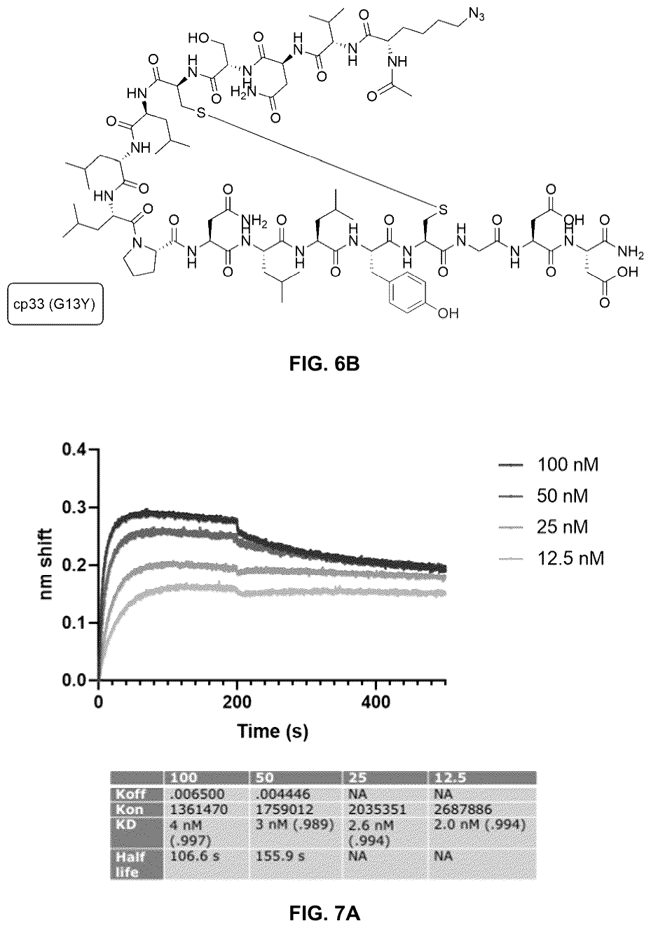

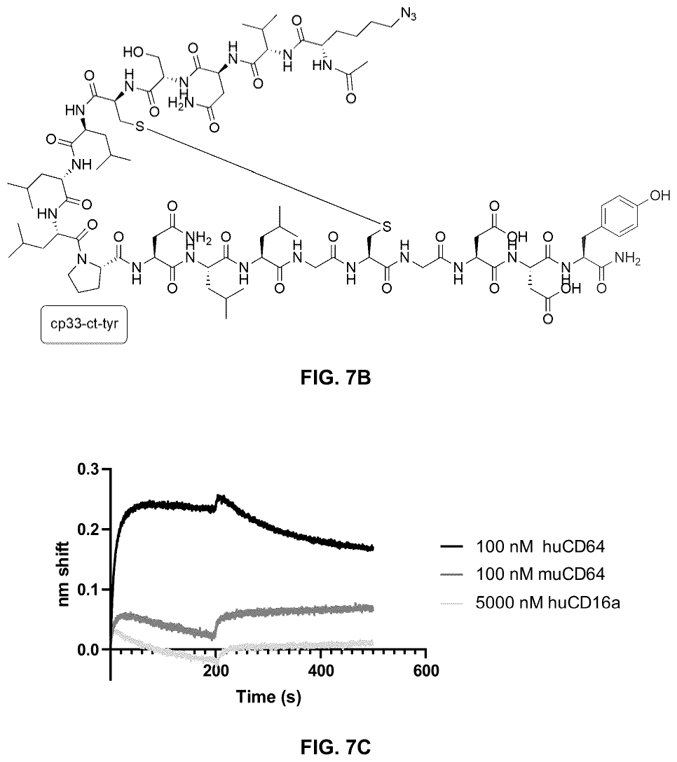

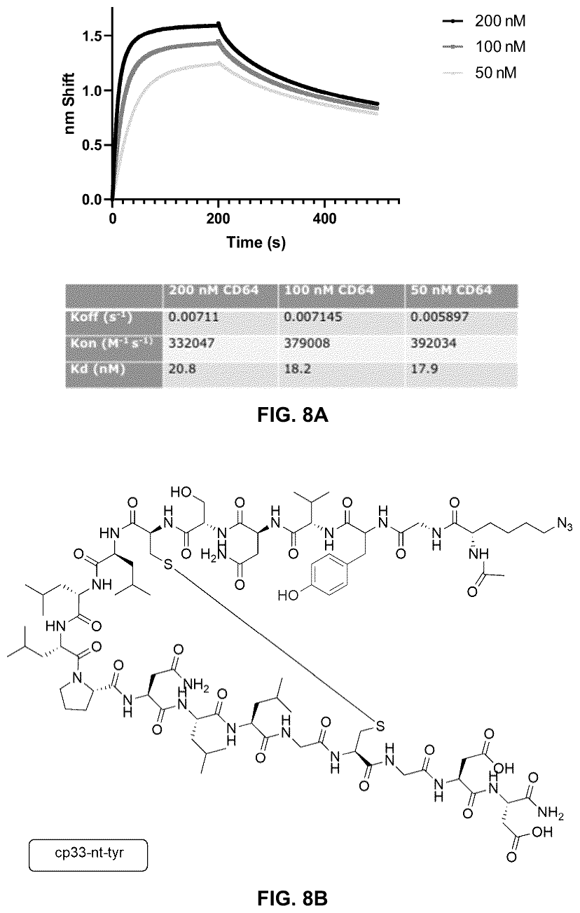

First CP33 peptide and various tyrosine mutants including the computationally predicted N-terminal mutant were synthesised by conventional solid phase peptide synthesis. These were synthesized to characterize CD64 binding affinity upon a) installation of a linker to enable for down-stream attachment of tumor antigen binding molecules (e.g. glutamate urea against PSMA, FIG. 3) and b) insertion of tyrosine mutations to enable downstream incorporation of SuFEx chemistry (FIG. 3). Binding affinity was assessed by BLI where streptavidin coated biosensor probes were loaded with biotin-PEG8-CP33 conjugates followed by addition of recombinant CD64. Four initial CP33-biotin linker conjugates were evaluated in BLI: a) the native CP33 peptide connected through the solvent exposed N terminus to a biotin-PEG linker, using SPAAC click chemistry (strained alkyne and azide shown pre-coupling rxn), b) a C term tyrosine mutant analog, c) an N-term tyrosine mutant analog, and d) an internal tyrosine mutant analog (G13Y). Respective structures and binding data are shown in FIGS. 4-7.

### Example 2. Characterization of CD64 Binding, Ternary Complex Formation, and Activation of Phagocytic Activity

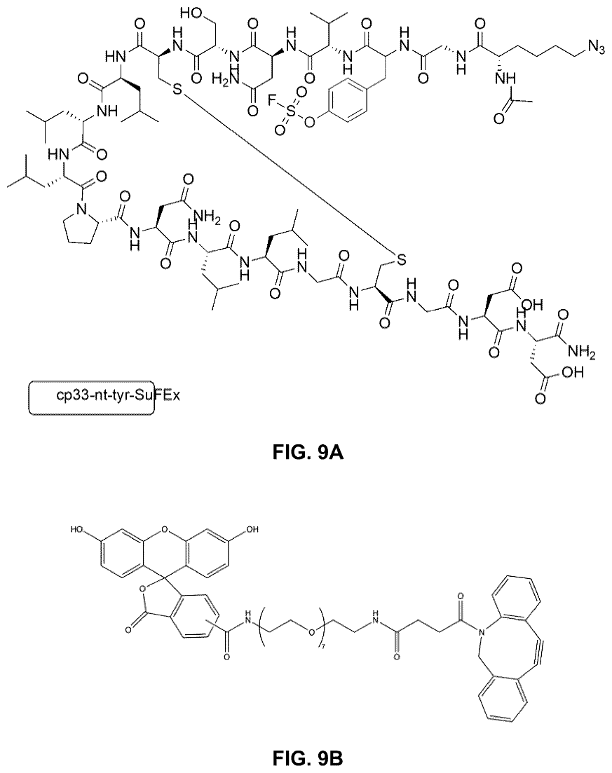

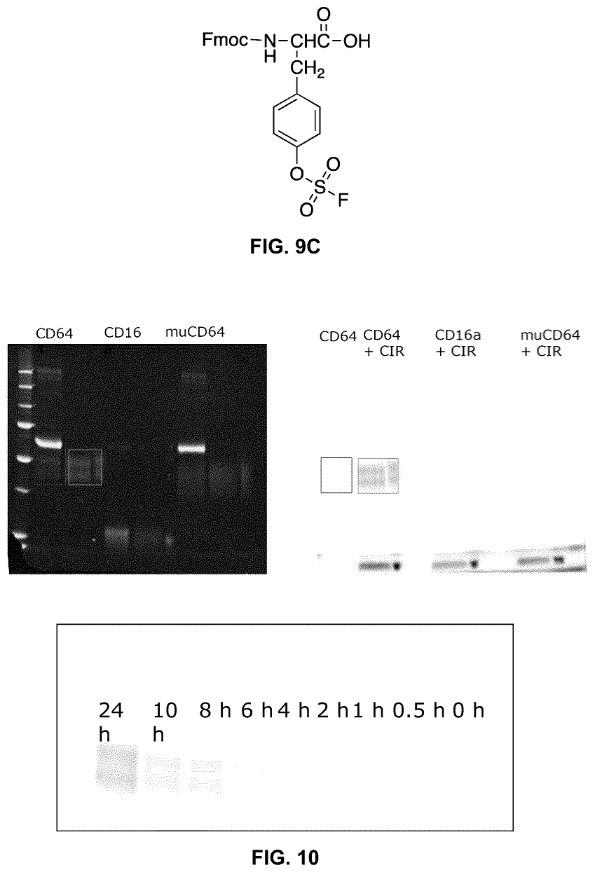

The results of binding assays enabled the following conclusions: 1) CP33 peptide conjugates can bind CD64 with high affinity and selectivity while engineered with a clickable linker for downstream tumor binding applications; 2) CD64 binding is sensitive to the position of tyrosine incorporation, with N term insertion generally most tolerated/least perturbing. The CP33(Nterm) tyrosine insertion mutant was subsequently engineered to contain SuFEx covalent binding chemistry to form CP33-Nterm-SuFEx. This lead CIR fragment was evaluated in CD64 kinetic covalent binding assays monitored by fluorescence SDS PAGE. Lead covalent peptide derivative CP33-Nterm-SuFEx was synthesized by solid phase peptide synthesis where the SuFEx was installed by one of two methods: a) as a pre-synthesized Fmoc tyr-OSO2F-COOH amino acid incorporated via SPPS (FIG. 9), or via treatment of a selectively deprotected tyrosine on the solid support resin, with AISF sulfonylation reagent. Following global peptide deprotection from solid phase peptide synthesis resin, a fluorescein-DBCO fragment was clicked to the CP33 fragment to generate the CIR fragment CP33-Nterm-SuFEx-linker-fluorescein (FIG. 9). This CIR fragment was incubated with CD64 for different amounts of time, where the fraction of covalent bound CD64 product (CIR-CD64) can be detected by fluorescence SDS-PAGE imaging. Notably, only covalently bound CIR fragment will remain bound to CD64 during SDS-PAGE giving rise to fluorescent bands. In these assays selective covalent binding of CIR fragment to CD64 was observed to take place on the order of hours. Importantly, no fluorescence labeling of related FcRs was observed.

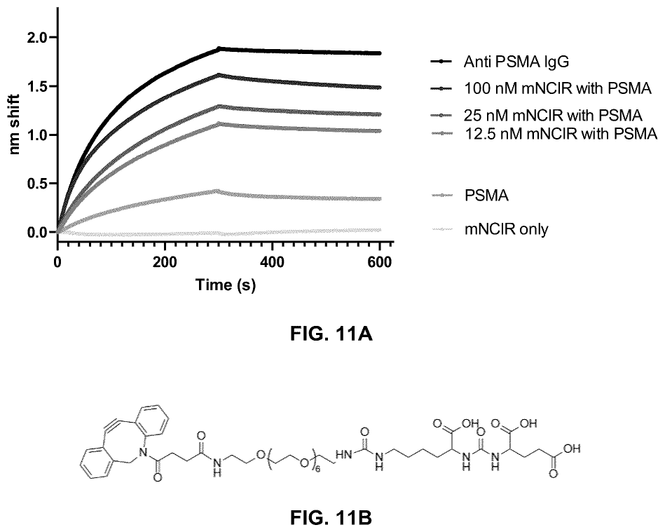

Next, the ability for the CIR to “bridge” the two receptors, (CD64 and PSMA), to form a ‘ternary complex’ (CD64:NCIR:PSMA) was tested using BLI (FIG. 11). This is important for CIR and NCIR function. This ability was tested by functionalizing a streptavidin biosensor with biotinylated Human CD64 and placing NCIRs with PSMA Receptor in solution. An increase in signal is associated with the PSMA receptor localizing to the CD64, through the bridging of the NCIR. This NCIR was created though an analogous fashion to the biotin and fluorescein derivatives using a DBCO-Peg7-GUL. Glutamate Urea Lysine (GUL) is a known binder of PSMA, an overexpressed cancer antigen on prostate cancer cells.

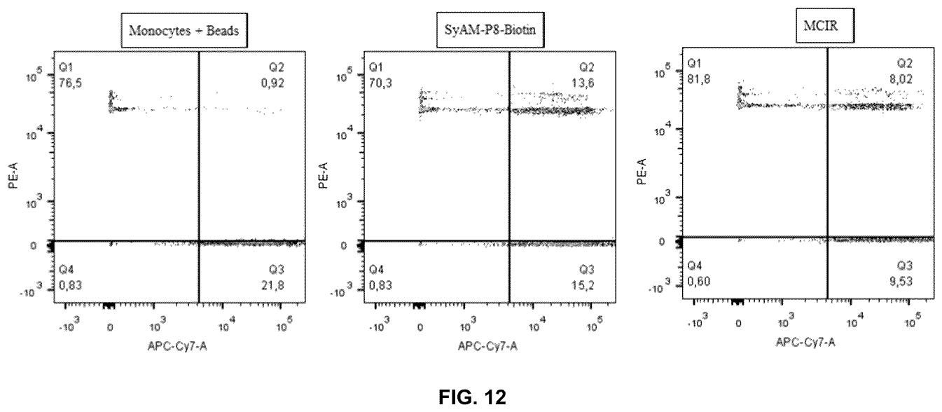

Building off the BLI ternary complex assay, NCIRs and CIRs were tested in a Flow cytometry based phagocytosis assay. Here YG Streptavidin beads (detected in PE channel) were incubated in the presence of coloured IFNγ activated U937 monocytes (detected in APC-Cy7 channel) within the presence of the CIR-Biotin. The ability of CIR-Biotin to redirect the monocytes to the Streptavidin beads is indicated by the appearance of a population containing both quarters (Q2) (FIG. 12).

As shown in FIGS. 11 and 12, CIRs and NCIR are able to ‘bridge’ or form a ternary complex, between CD64 and Streptavidin or PSMA, with either the receptor alone or on the surface of a cell.

### Example 3. Characterization of Sulfonyl Fluoride Covalent Binding Groups

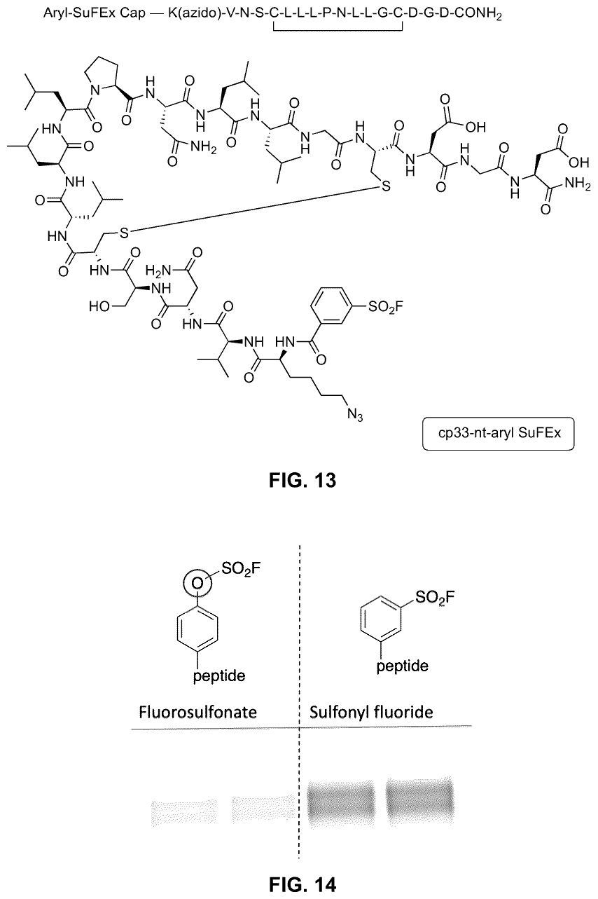

CIR containing alternative covalent labelling domains were explored (FIG. 13). A sulfonyl fluoride labelling chemistry was used as opposed to the fluorsulfonate (FIG. 14). Upon running a similar SDS-PAGE gel as in FIG. 9, it was observed that the sulfonyl fluoride chemistry has faster labelling kinetics then the previous CIR (FIG. 14).

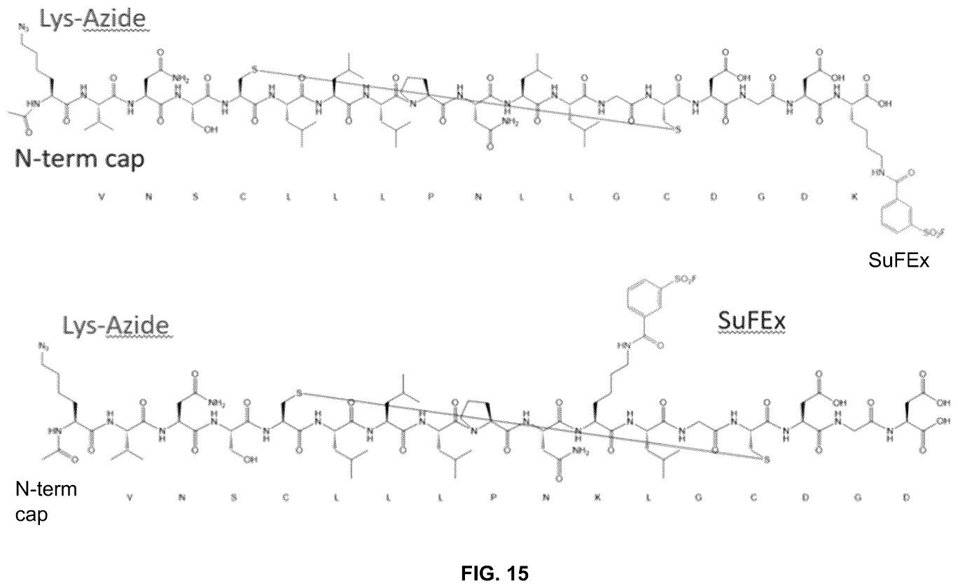

Variations of the sulfonyl fluoride CIR were synthesized with the sulfonyl fluoride labelling group at different locations within the cP33 peptide (FIG. 13, FIG. 15). Three variations were synthesized: one with the sulfonyl fluoride group at the C-terminus (cP33 ct-aryl SuFEx; SEQ ID NO: 12), one with the sulfonyl fluoride at an internal position replacing leucine at position 7 within the peptide (cp33 int-aryl SuFEx; SEQ ID NO: 14) and one with the sulfonyl fluoride at the n-terminus (cp33 nt-aryl SuFEx; SEQ ID NO: 10). The C-terminus and internal variations were synthesized by incorporating a Lys-DDE amino acid at the corresponding position using traditional F-moc based solid phase peptide synthesis (SPPS). The DDE group is deprotected by 2% hydrazine after the entire peptide is synthesized and the aryl SuFEx is coupled onto the free amine of the lysine on resin using 3-(fluorosulfonyl)benzoic acid. Biotinylated and fluorescent versions of these CIRs were synthesized through SPAAC click chemistry by reacting DBCO-biotin or DBCO-A647, respectively, onto the azide at the n-terminus of the peptide using the same procedure as previously described.

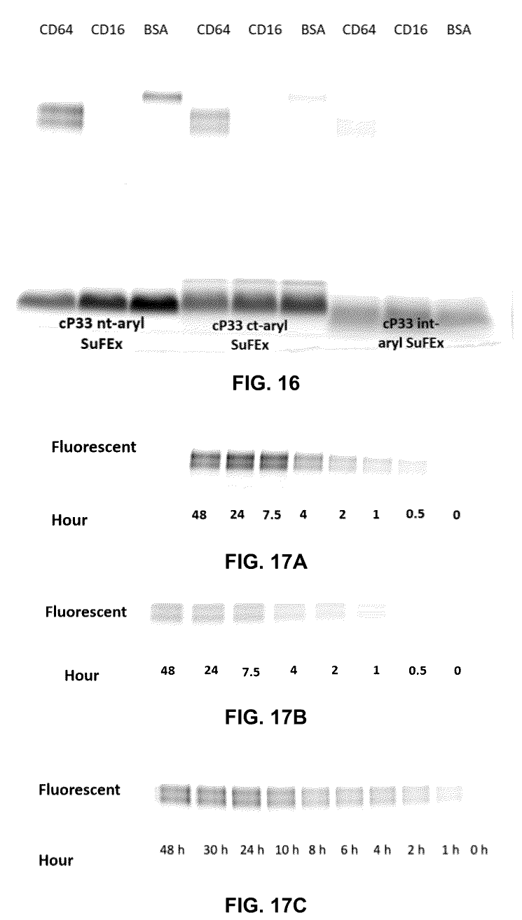

The specificity of the three sulfonyl fluoride CIR variations for labelling CD64 were investigated using a fluorescent SDS-PAGE assay similar to as described above (FIG. 16). 1.6 μM CIR clicked to A647 fluorescent dye was incubated with 800 nM of protein for 7 hours at room temperature in the dark. Three different proteins were assayed to test specificity: CD64, CD16, and BSA. The samples were then run on a 4-20% gradient acrylamide gel and fluorescence was measured using the Cy5 channel with an Amersham Typhoon biomolecular imager. Only covalently labelled CIR will remain bound to CD64 in SDS-PAGE giving rise to fluorescent bands.

The darkness of the bands represents covalent CIR binding. All three sulfonyl fluoride variants showed great preferential labelling for CD64 (dark band) compared to related FcRs such as CD16 (very light band). Notably, cP33-ct-aryl SuFEx showed greater binding selectivity compared to the other variants as proportionally less BSA was covalently labelled compared to CD64.

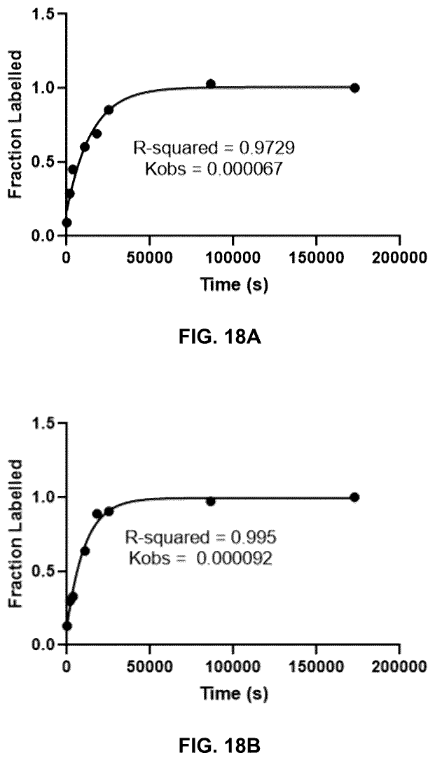

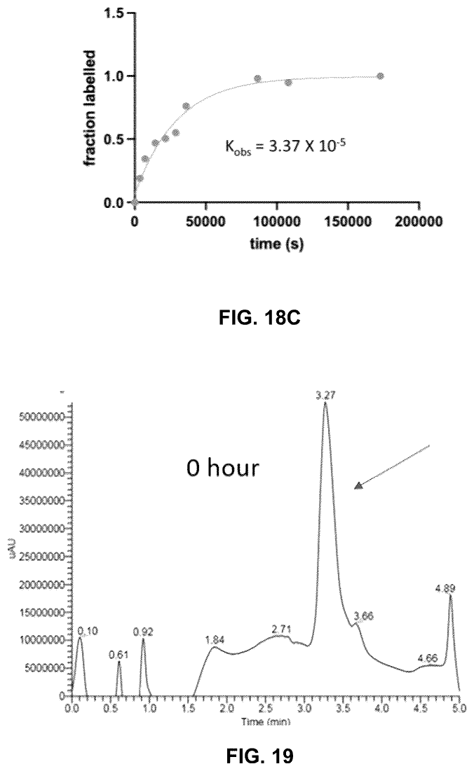

The kinetics of the labelling reaction was also investigated using fluorescent SDS-PAGE gels similar to as described above. 1.6 μM of CIR was incubated with 800 nM CD64 for different amounts of time before being run on a SDS-PAGE gel (FIG. 17). The fluorescence intensity of each band was then measured and then compared to the fluorescence intensity of the band at 100% fraction labelled (where the fluorescence intensity reaches a plateau) to make a fraction bound vs time graph (FIG. 18). The curve of the graph was fitted to a one-phase association curve to find the K_{obs}.

The labelling rate for all three variants were similar, with cP33 nt-aryl SuFEx having the slowest labelling with a K_{obs} of 3.37×10⁻⁵ s⁻¹. cP33 int-aryl SuFEx has the fastest labelling rate with a K_{obs} of 9.2×10⁻⁵ s⁻¹. cP33 ct-aryl SuFEx has a K_{obs} of 6.7×10⁻⁵ s⁻¹. All three CIR variants showed decent covalent labelling rates with complete labelling seen by ˜8 hours at room temperature.

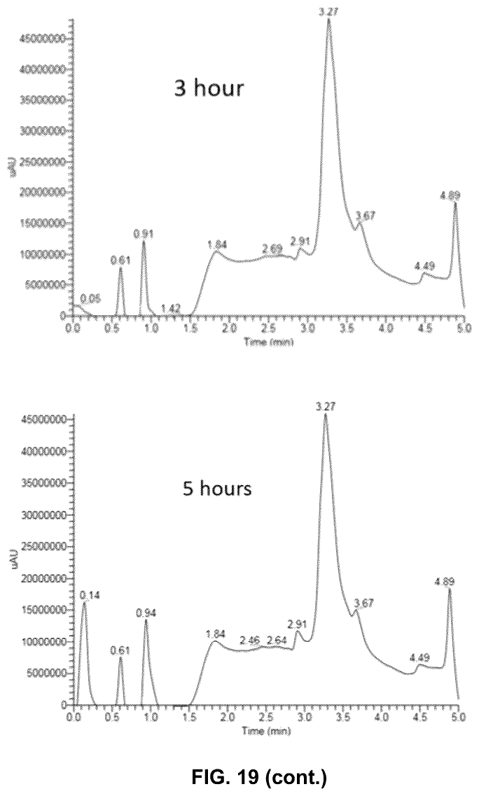

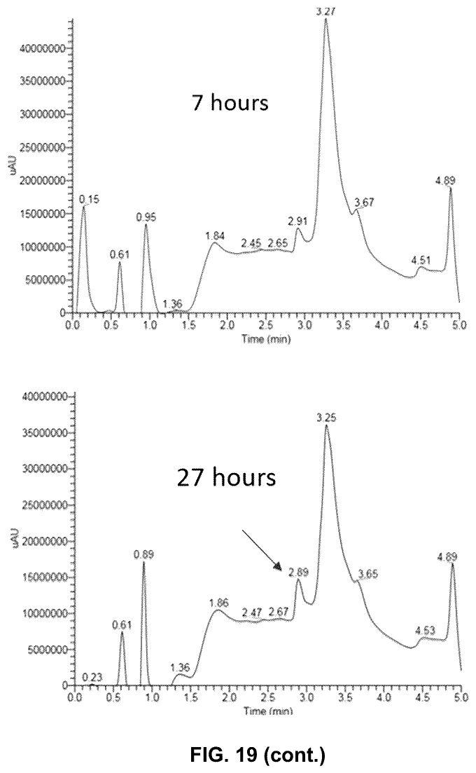

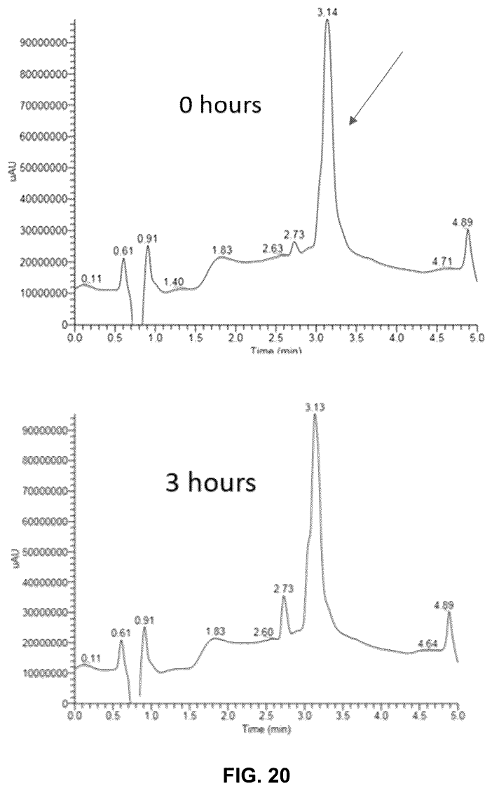

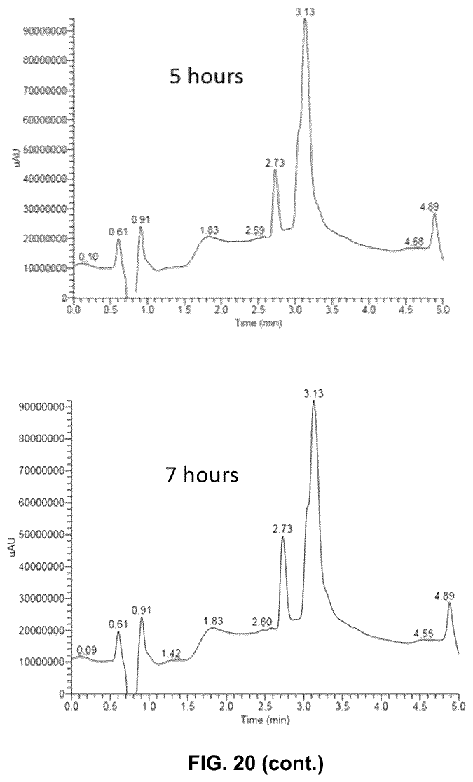

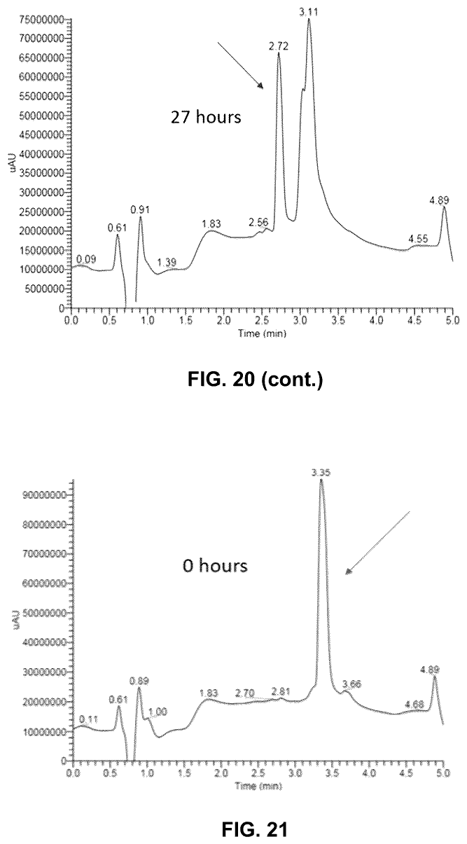

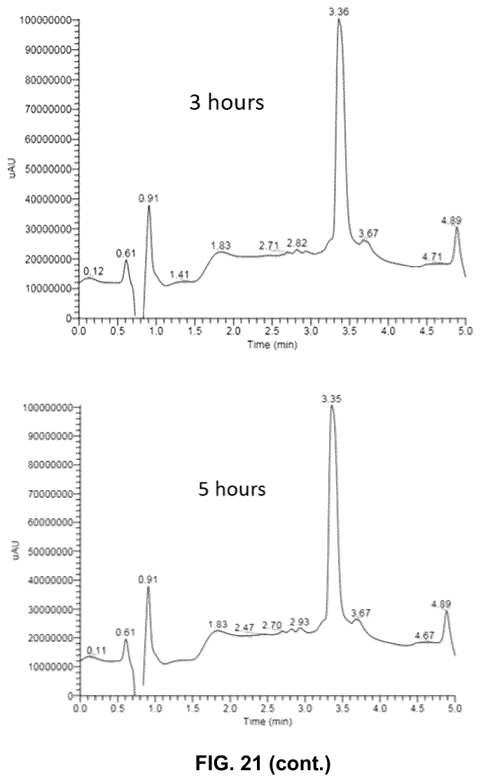

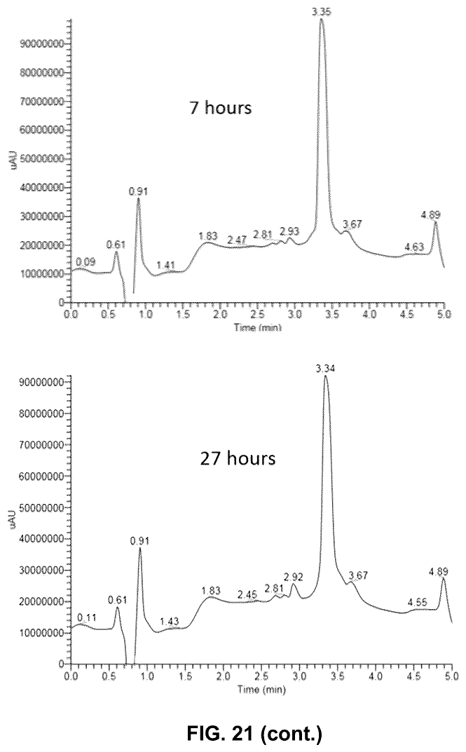

The stability of the three sulfonyl fluoride variants in aqueous conditions at room temperature were then assessed using LC-MS. LC-MS measurements were taken over 27 hours and peaks on the UV chromatogram were monitored for CIR degradation (FIG. 19-21).

All three sulfonyl fluoride variants showed good stability in aqueous conditions over 27 hours. All three had strong base peaks that remained present over 27 hours. cP33-nt-aryl SuFEx showed no degradation peaks while cP33-ct-aryl SuFEx showed some slight degradation peaks by 27 hours. cP33-int-aryl SuFEx was the most unstable and showed noticeable degradation peaks by 27 hours, however the presence of a strong base (undegraded) peak was still detected even at 27 hours.

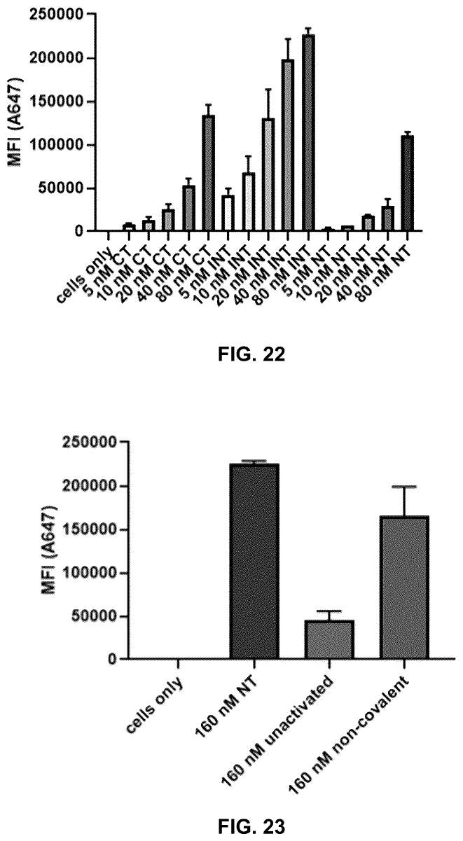

The ability of the three sulfonyl fluoride CIRs to bind CD64 on the immune cell surface was then assessed using flow cytometry (FIG. 22). U937 cells were fixed using paraformaldehyde 24 hours after being activated with IFNγ. The fixed cells were then incubated with different concentrations of CIR that had been clicked to an A647 fluorescent handle for 7 hours at room temperature. U937 cells that were not activated with IFNγ and therefore should have a much lower level of CD64 expression were also assayed as a control (FIG. 23). The mean fluorescence intensity at the Cy5 channel was measured using a BD LSRII flow cytometer.

All three sulfonyl fluoride CIR variants were shown to specifically and effectively label CD64 on the surface of fixed cells, with MFI signals above the cells only negative control. FIG. 22 shows the CIR concentration dependence of covalent labelling of CD64 as MFI increases with CIR concentration. Notably, MFI decreases when using U937 cells that were not activated and have a lower CD64 expression level. This CD64 dependence shows that the CIRs bind specifically to CD64. The non-covalent analog shows less covalent labeling compared to the covalent version, showing the importance of covalency.

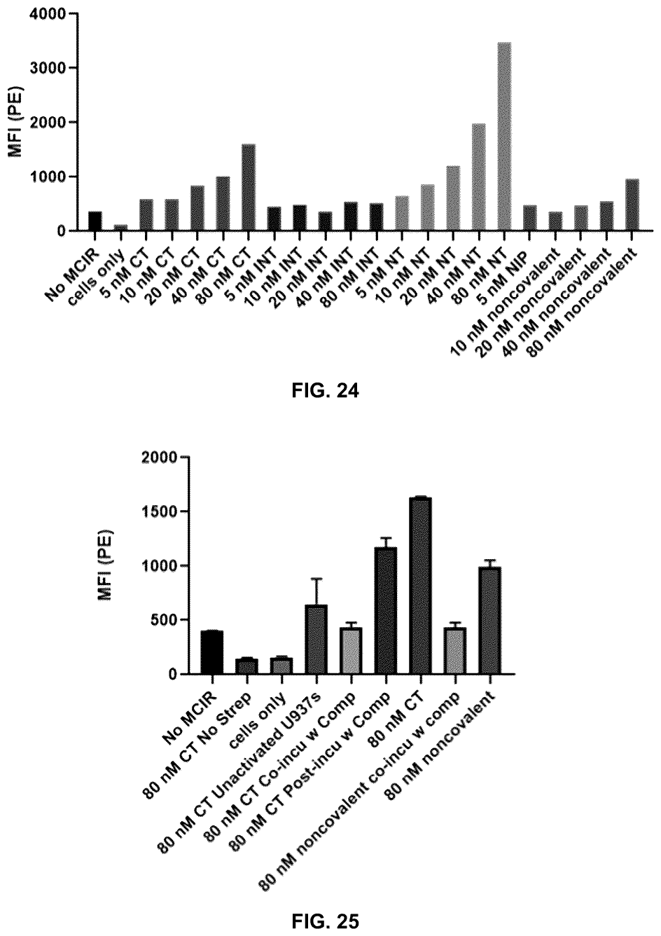

The ability of the three sulfonyl fluoride CIR variants to label CD64 on the surface of live cells was assayed using CIRs clicked to a biotin handle. CIR-biotin was incubated with U937 cells for 24 hours at 37° C. along with IFNγ. Post reaction, the cells were washed and incubated with streptavidin-PE. The streptavidin binds tightly to the biotin on the CIR and the fluorescent PE signal was read using a BD LSRII flow cytometer (FIG. 24). 100X CIR not labelled with biotin was used as competitor control to assess specificity and covalency (FIG. 25).

cP33-ct-aryl SuFex and cP33-nt-aryl SuFEx both showed specific and effective covalent labelling of CD64 on the surface of live cells. They showed a significant increase in labeling signal over the “no MCIR” control, demonstrating that the fluorescent signal measured is not from internalization of the streptavidin-PE. Further, they showed a greater covalent labelling over the non-covalent analog, illustrating the importance of covalency for this platform (FIG. 25). Notably, the fact that streptavidin-PE was able to label the CIRs on the surface of the cell after 24 hours proves that the CIRs are not internalized within the cell during the labeling process and can effectively label the surface of the cell. MFI decreases when using CD64 cells that were not activated and have a lower CD64 expression level. This CD64 dependence suggests that the CIRs bind specifically to CD64. Competitor co-incubated with CIR and the non-covalent analog showed disruption of labelling (decreased MFI) whereas competitor spiked post CIR incubation showed no disruption of labelling showing the covalency of binding.

### Example 4. Further Characterization of CIR CD64 Labelling, Immune Cell Activation and Tumour Targeting Ability

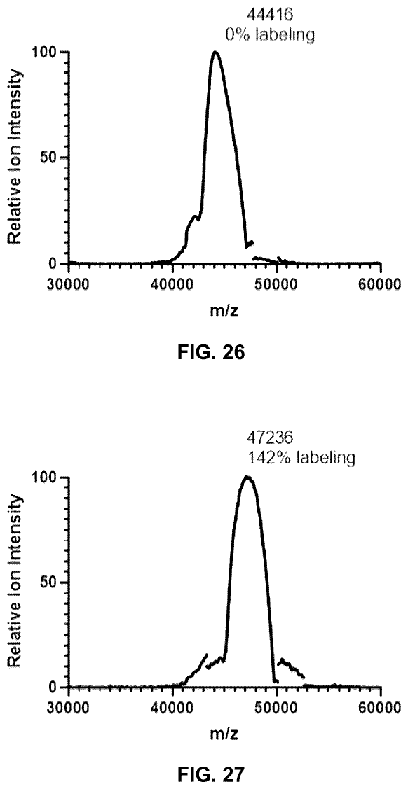

The ability of the CIR (cP33-nt-aryl SuFEx) to covalently label CD64 in solution was further verified using matrix-assisted laser desorption/ionization mass spectrometry (MALDI). FIG. 26 represents the MALDI peak for unlabeled CD64 showing a mass peak at 44416 m/z. FIG. 27 represents the MALDI peak when 5.4 μM CD64 was incubated with 50 μM CIR in PBS for 24 hours. The excess unreacted CIR and salt was removed using Zeba Spin Desalting Column 7K MWCO (Thermo Fisher, cat number 89882). The mass peak in FIG. 27 shifted to increase to 47236 m/z corresponding to the increased mass of the CIR covalently labeled to the CD64.

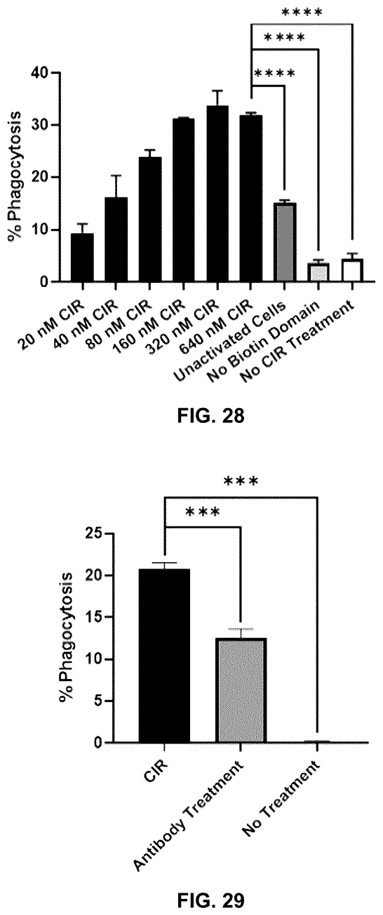

The ability of the CIR to direct immune cell phagocytic activity was further investigated using a modified 2-colour phagocytosis assay (FIG. 28). In this assay, varying concentrations of CIR-biotin were incubated with IFNγ activated U937 monocytes for 24 hours. The cells were then dyed using a fluorescent lipophilic cell membrane dye and incubated with YG fluorescent streptavidin labeled beads for 1 hour at 37 degrees Celsius. Fluorescence was measured using flow cytometry. Successful induced phagocytosis of the target bead by the monocyte is determined by looking at the population that is double positive for both the fluorescent signal of the monocytes and the fluorescent signal of the bead. Percent phagocytosis was calculated by comparing the ratio of successful phagocytosis events to the total number of monocytes. FIG. 28 shows a dose-dependent increase in induced phagocytosis as CIR concentration increases. Further, unactivated cells that have less CD64 expression that were incubated with 640 nM CIR had less phagocytosis showing that the CIR-induced phagocytosis is CD64 specific. When cells were incubated with 640 nM CIR that did not have the biotin targeting domain, no phagocytosis was induced showing that CIR is target specific and does not induce a non-specific immune response.

The above-described phagocytosis assay was repeated to compare the efficacy of the CIR to an analogous antibody treatment (FIG. 29). Fluorescently dyed IFNγ activated U937 monocytes were incubated with 100 nM CIR-biotin or an anti-DNP antibody (SPE7) functionalized with a biotin for 1 hour as described in Kapcan E, et al., Covalent Stabilization of Antibody Recruitment Enhances Immune Recognition of Cancer Targets. Biochemistry. 2021 May 18;60(19):1447-1458, herein incorporated by reference. YG fluorescent streptavidin labeled beads were then incubated with the monocytes for 1 hour before fluorescence was read using flow cytometry and % phagocytosis was calculated as described previously. FIG. 29 shows that the CIR induced more phagocytosis compared to the antibody treatment.

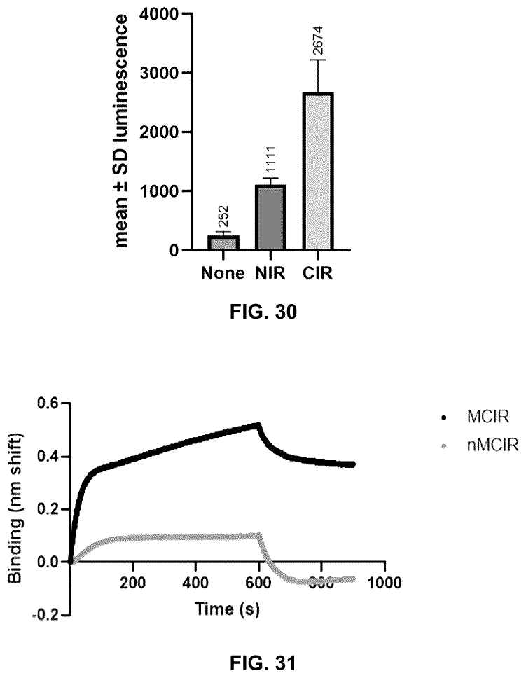

The ability for the CIR to activate immune cells was investigated using a THP1 NF-kB-Luc2 reporter cell line (ATCC, TIB-202-NFKB-LUC2) which produces luciferase when the NF-kB activation pathway is stimulated (FIG. 30). Streptavidin labeled beads were labeled with CIR-biotin or the analogous non-covalent version (NIR) and incubated with the THP1 NF-kB-Luc2 reporter cell for 24 hours in a 96-well microplate. Luminescence was then measured using a microplate reader (Tecan SPARK multimode microplate reader). Cells incubated with CIR showed a significant increase in luminescence production compared to no treatment showing that CIRs can induce immune cell activation (FIG. 30). Further, CIRs show a significant increase in luminescence production compared to the non-covalent analog, showing that covalency increases immune cell activation efficacy (FIG. 30).

A version of CIR was synthesized which can bind and target a tumour marker receptor urokinase-type plasminogen activator receptor (uPAR) by clicking cP33-nt-aryl SuFEx to a modified uPAR binding peptide ligand (uPAR MCIR).

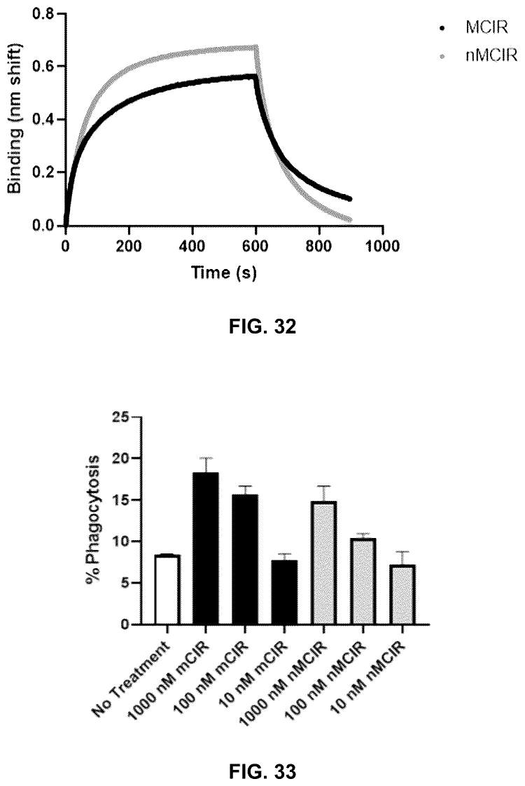

The ability of the uPAR MCIR to form a ternary complex and bridge CD64 and uPAR was assessed using BLI (FIG. 31 and FIG. 32). This ability was first tested by functionalizing a streptavidin biosensor with 100 nM biotinylated human CD64, which is then dipped into a pre-equilibrated solution of 500 nM human uPAR and 500 nM of either uPAR MCIR or the analogous non-covalent version (nMCIR). An increase in signal is associated with the uPAR localizing to the CD64, through the bridging of the MCIR/nMCIR. FIG. 31 shows that MCIR can successfully form a ternary complex with CD64 and uPAR when the MCIR first binds uPAR, suggesting that covalency may increase ternary complex formation efficacy.

Further, a reciprocal assay was performed in which a streptavidin biosensor functionalized with biotinylated human uPAR was dipped into a pre-equilibrated solution of human CD64 and either uPAR MCIR or nMCIR (FIG. 32). An increase in signal is associated with the CD64 localizing to the uPAR, through the bridging of the MCIR/nMCIR. FIG. 32 shows that both MCIR and nMCIR can successfully form a ternary complex with CD64 and uPAR when the MCIR first binds CD64.

The previously described phagocytosis assay was repeated to assess the efficacy of the uPAR MCIR (FIG. 33). Fluorescently dyed IFNγ activated U937 monocytes were incubated with the indicated concentrations of either uPAR MCIR (mCIR) or the non-covalent version (nMCIR). YG fluorescent streptavidin labeled beads were labelled with biotinylated human uPAR and were then incubated with the monocytes for 1 hour. Fluorescence was then read using flow cytometry and % phagocytosis was calculated as described previously. FIG. 33 shows that both uPAR MCIR and uPAR nMCIR can induce an increase in % phagocytosis of uPAR-labeled targets at the concentrations used in this assay compared to no treatment, demonstrating that proximity induction occurs in this model system.

### Example 5. Materials and Methods for Producing CIRs

### Biotinylation Procedures

DBCO-peg4-biotin was obtained from BroadPharm (13.3 mM stock solution).

All reactions were performed so that the biotin was the limiting reagent, either by using less volume of a 100 uM stock (cp33, cp33 (C-term tyr), and cp33-nt) or by using a lower concentration of the biotin in the final solution (cp33-Nterm-SuFEx)

### Cp33 (Original Sequence) Biotinylation

2.3 uL of an 8.664 mM cp33 stock solution+197.7 uL PBS

1.35 uL of 13.3 mM DBCO-peg4-biotin solution+178.65 uL of PBS

Added both mixtures together and left O/N to stir

Cp33 (C-Term tyr) Biotinylation

1.6 uL of a 12.5 mM cp33-ct stock+198.4 uL PBS

1.35 uL of 13.3 mM DBCO-peg4-biotin+178.67 uL PBS

Combined both solutions and left O/N to react

### Cp33-nt Biotinylation

3.44 uL of 5.82 mM cp33-nt stock+196.56 uL PBS

1.35 uL of 13.3 mM DBCO biotin, +178.67 uL PBS

Combined both solutions and leave O/N stirring

Final Concentrations of Reagents in 380 uL Solution (cp33, cp33 (C-Term tyr), and cp33-nt)

Peptide ˜53 uM

Biotin ˜47 uM

### Cp33-nt-SuFEx Biotinylation

4 uL of a 5 mM CP33-nt-SuFEx stock+1.35 uL of 13.3 mM DBCO-biotin+194.65 uL PBS

Increased concentration in solution by decreasing reaction volume, left for 3 hr before putting into the −80→full conversion with a higher concentration

Performed twice with 100% conversion

Final Concentrations of Reagents in 200 uL Solution (cp33 nt-SuFEx)

Peptide 100 uM

Biotin 90 uM

### Fluorescein Procedure

Using a fluorescein-peg8-DBCO stock

10 mM stock dissolved in DMSO

All vials were covered with tinfoil over the course of the reaction

All the fluor reactions were performed with equimolar fluorescein and peptide→so far this has seen 100% conversion with no detectable starting material

### Cp33-nt

3.44 uL of 5.82 mM cp33-nt+2 uL of 10 mM fluor DBCO+194.56 uL PBS

Left to react for 3 hr before removing and putting into −80

### Cp33-nt SuFEx

4 uL of 5 mM cp33-nt-SuFEx+2 uL of 10 mM fluor-DBCO+194 uL PBS

Left to react for 3 hr before putting into the −80

### Final Concentrations of Reagents in Solution

100 uM of both fluorescein and peptide

### GUL-Peg7-DBCO Click Procedure

Use a DBCO-peg7-GUL

1.25 mM stock solution

### Cp33-nt (Original Sequence)

5.48 uL of an 3.65 mM cp33 stock solution+194.5 uL PBS 16 uL of 1.25 mM DBCO-peg4-biotin solution+184 uL of PBS

Added both mixtures together and left O/N to stir

uPAR MCIR Click Procedure

100 μM cP33-nt stock and 100 μM DBCO-modified synthetic uPAR binding peptide ligand (SEQ ID NO: 16) in PBS for 3 hours stirring at room temperature

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

Sequences
Native CP33 peptide sequence
(SEQ ID NO: 1):
VNSCLLLPNLLGCGDD
Disulfide bond between C4 and C13
C-terminal amidation (-CONH2) may be present
or absent
CP33 peptide sequence variant
(SEQ ID NO: 2):
VNSCLLLPNLLGCDGD
Disulfide bond between C4 and C13
C-terminal amidation present or absent
CP33 C-term tyrosine peptide sequence
(SEQ ID NO: 3):
XVNSCLLLPNLLGCGDDY
X is K, Ac-K(azido), or absent
Disulfide bond between C5 and C14
C-terminal amidation present or absent
CP33 C-term tyrosine peptide sequence
(SEQ ID NO: 4):
XVNSCLLLPNLLGCDGDY
X is K, Ac-K(azido), or absent
Disulfide bond between C5 and C14
C-terminal amidation present or absent
CP33 internal tyrosine (G13Y) mutant peptide
sequence
(SEQ ID NO: 5):
XVNSCLLLPNLLYCGDD
X is K, Ac-K(azido), or absent
Disulfide bond between C5 and C14
C-terminal amidation present or absent
CP33 internal tyrosine (G13Y) mutant peptide
sequence
(SEQ ID NO: 6):
XVNSCLLLPNLLYCDGD
X is K, Ac-K(azido), or absent
Disulfide bond between C5 and C14
C-terminal amidation present or absent
CP33 N-term tyrosine peptide sequence
(SEQ ID NO: 7):
XGYVNSCLLLPNLLGCGDD
X is K, Ac-K(azido), or absent
Disulfide bond between C7 and C16
C-terminal amidation present or absent
CP33 N-term tyrosine peptide sequence
(SEQ ID NO: 8):
XGYVNSCLLLPNLLGCDGD
X is K, Ac-K(azido), or absent
Disulfide bond between C7 and C16
C-terminal amidation present or absent
CP33-nt-aryl SuFEx peptide sequence
(SEQ ID NO: 9):
XVNSCLLLPNLLGCGDD
X is K or Aryl-SuFEx cap-K(azido)
Disulfide bond between C5 and C14
C-terminal amidation present or absent
CP33-nt-aryl SuFEx peptide sequence
(SEQ ID NO: 10):
XVNSCLLLPNLLGCDGD
X is K or Aryl-SuFEx cap-K(azido)
Disulfide bond between C5 and C14
C-terminal amidation present or absent
CP33-ct-aryl SuFEx peptide sequence
(SEQ ID NO: 11):
XVNSCLLLPNLLGCGDDX
X1 is K or K(azido)
X18 is K or K-SuFEx
Disulfide bond between C5 and C14
CP33-ct-aryl SuFEx peptide sequence
(SEQ ID NO: 12):
XVNSCLLLPNLLGCDGDX
X1 is K or K(azido)
X18 is K or K-SuFEx
Disulfide bond between C5 and C14
CP33-int-aryl SuFEx peptide sequence
(SEQ ID NO: 13):
XVNSCLLLPNXLGCGDD
X1 is K or K(azido)
X11 is K or K-SuFEx
Disulfide bond between C5 and C14
CP33-int-aryl SuFEx peptide sequence
(SEQ ID NO: 14):
XVNSCLLLPNXLGCDGD
X1 is K or K(azido)
X11 is K or K-SuFEx
Disulfide bond between C5 and C14
Synthetic uPAR binding peptide
ligand
(SEQ ID NO: 15)
L-Lys-Gly-Gly-L-Ser-Gly-L-Asp-L-Cha-L-
Phe-D-Ser-D-Arg-L-Tyr-L-Leu-L-Trp-L-Ser
Modified synthetic uPAR binding
peptide ligand
(SEQ ID NO: 16)
L-Lys-Gly-Gly-L-Ser-Gly-L-Asp-L-Cha-L-
Phe-D-Ser-D-Arg-L-Ala-L-Leu-L-Trp-L-Ser
CP33 C-term tyrosine peptide sequence
(SEQ ID NO: 17)
AQVNSCLLLPNLLGCGDDY
Disulfide bond between C6 and C15

1. A covalent immune recruiter (CIR) comprising an Fc receptor (FcR) targeting domain (FTD), a covalent binding group (CBG), and a first target binding domain (TBD), wherein the FTD specifically binds an FcR on an immune cell, and wherein the CBG comprises a functional group that, on binding of the FTD to the FcR, forms a covalent bond with an amino acid in the FcR.

2. The CIR of claim 1, wherein the FcR is an FcγR, optionally selected from CD64, CD32, CD16a, and CD16b, optionally human CD64.

3. The CIR of claim 1, wherein the FTD comprises a peptide having the sequence of SEQ ID NO: 1 or a functional variant thereof or SEQ ID NO: 2 or a functional variant thereof.

4. The CIR of claim 3, wherein the FTD comprises a peptide having the sequence of SEQ ID NO: 3, SEQ ID NO: 4, SEQ ID NO: 7, SEQ ID NO: 8, SEQ ID NO: 9, SEQ ID NO: 10, SEQ ID NO: 11, or SEQ ID NO: 12, or functional variants thereof.

5. The CIR of claim 1, wherein the TBD binds a protein that is overexpressed in a disease, disorder or condition, optionally cancer.

6. The CIR of claim 1, wherein the TBD comprises glutamate urea lysine (GUL).

7. The CIR of claim 1, wherein the TBD comprises a uPAR binding ligand, optionally a synthetic uPAR binding peptide ligand, optionally comprising a sequence of SEQ ID NO: 15 or SEQ ID NO: 16 or functional variants thereof.

8. The CIR of claim 1, wherein the TBD comprises biotin.

9. The CIR of claim 1, wherein the CBG comprises SuFEx, optionally fluorosulfate, fluorosulfonate, or sulfonyl fluoride.

10. The CIR of claim 1, further comprising a second TBD, optionally comprising a third TBD.

11. A functionalized cell modified by the CIR of claim 1.

12. The cell of claim 11, wherein the cell is selected from lymphocytes, monocytes, macrophages, optionally tumor-associated macrophages, polymorphonuclear cells, erythrocytes and megakaryocytes, B cells, NK cells, neutrophils, basophils, eosinophils, and dendritic cells.

13. The cell of claim 11, wherein the FTD comprises a peptide having the sequence of SEQ ID NO: 1 or a functional variant thereof or SEQ ID NO: 2 or a functional variant thereof, optionally SEQ ID NO: 3, SEQ ID NO: 4, SEQ ID NO: 7, SEQ ID NO: 8, SEQ ID NO: 9, SEQ ID NO: 10, SEQ ID NO: 11, or SEQ ID NO: 12, or functional variants thereof.

14. A method of generating a functionalized cell, the method comprising:
providing a cell comprising an FcR; and
contacting the cell with the CIR of claim 1.

15. A method of treating or preventing a disease, disorder or condition that is treatable or preventable by immunotherapy, comprising administering a therapeutically effective amount of
a) a CIR according to claim 1; or
b) a functionalized cell comprising the CIR;
to a subject in need thereof.

16. The method of claim 15, wherein the FTD comprises a peptide having the sequence of SEQ ID NO: 1 or a functional variant thereof or SEQ ID NO: 2 or a functional variant thereof, optionally SEQ ID NO: 3, SEQ ID NO: 4, SEQ ID NO: 7, SEQ ID NO: 8, SEQ ID NO: 9, SEQ ID NO: 10, SEQ ID NO: 11, or SEQ ID NO: 12, or functional variants thereof.

17. The method of claim 15, wherein the disease, disorder, or condition that is treatable by immunotherapy is a cancer, an autoimmune disease, allergy, or transplant rejection.

18. The method of claim 15, wherein the cells are autologous.

19. The method of claim 15, wherein the cells are allogenic.

20. A kit comprising
a) an FcR targeting component comprising an FcR targeting domain, a covalent binding group, and an acceptor group for covalent attachment of a target binding domain and/or a target binding component comprising a target binding domain and a cognate donor group for covalent attachment to the acceptor group of the FcR targeting component;
b) a CIR according to claim 1; or
c) a functionalized cell comprising the CIR; and
one or more of a suitable container or packaging therefor and/or instructions for use thereof.


SEQUENCE LISTING (17 sequences)

SEQ ID NO: 1 - protein, 16 aa, synthetic construct
  DISULFID 4..13: note = Disulfide bond between C4 and C13
VNSCLLLPNLLGCGDD

SEQ ID NO: 2 - protein, 16 aa, synthetic construct
  DISULFID 4..13: note = Disulfide bond
VNSCLLLPNLLGCDGD

SEQ ID NO: 3 - protein, 18 aa, synthetic construct
  MOD_RES 1: note = Ac-K(azido)
  DISULFID 5..14: note = Disulfide bond between C5 and C14
  VARIANT 1: note = K, Ac-K(azido), or absent
XVNSCLLLPNLLGCGDDY

SEQ ID NO: 4 - protein, 18 aa, synthetic construct
  MOD_RES 1: note = Ac-K(azido)
  DISULFID 5..14: note = Disulfide bond between C5 and C14
  VARIANT 1: note = K, Ac-K(azido), or absent
XVNSCLLLPNLLGCDGDY

SEQ ID NO: 5 - protein, 17 aa, synthetic construct
  MOD_RES 1: note = Ac-K(azido)
  DISULFID 5..14: note = Disulfide bond between C5 and C14
  VARIANT 1: note = K, Ac-K(azido), or absent
XVNSCLLLPNLLYCGDD

SEQ ID NO: 6 - protein, 17 aa, synthetic construct
  MOD_RES 1: note = Ac-K(azido)
  DISULFID 5..14: note = Disulfide bond between C5 and C14
  VARIANT 1: note = K, Ac-K(azido), or absent
XVNSCLLLPNLLYCDGD

SEQ ID NO: 7 - protein, 19 aa, synthetic construct
  MOD_RES 1: note = Ac-K(azido)
  DISULFID 7..16: note = Disulfide bond between C7 and C16
  VARIANT 1: note = K, Ac-K(azido), or absent
XGYVNSCLLLPNLLGCGDD

SEQ ID NO: 8 - protein, 19 aa, synthetic construct
  MOD_RES 1: note = Ac-K(azido)
  DISULFID 7..16: note = Disulfide bond between C7 and C16
  VARIANT 1: note = K, Ac-K(azido), or absent
XGYVNSCLLLPNLLGCDGD

SEQ ID NO: 9 - protein, 17 aa, synthetic construct
  MOD_RES 1: note = Aryl-SuFEx cap-K(azido)
  DISULFID 5..14: note = Disulfide bond between C5 and C14
  VARIANT 1: note = K or Aryl-SuFEx cap-K(azido)
XVNSCLLLPNLLGCGDD

SEQ ID NO: 10 - protein, 17 aa, synthetic construct
  MOD_RES 1: note = Aryl-SuFEx cap-K(azido)
  DISULFID 5..14: note = Disulfide bond between C5 and C14
  VARIANT 1: note = K or Aryl-SuFEx cap-K(azido)
XVNSCLLLPNLLGCDGD

SEQ ID NO: 11 - protein, 18 aa, synthetic construct
  VARIANT 1: note = K or K(azido)
  MOD_RES 18: note = K-SuFEx
  DISULFID 6..14: note = Disulfide bond between C5 and C14
  MOD_RES 1: note = K(azido)
  VARIANT 18: note = K or K-SuFEx
XVNSCLLLPNLLGCGDDX

SEQ ID NO: 12 - protein, 18 aa, synthetic construct
  MOD_RES 1: note = K(azido)
  MOD_RES 18: note = K-SuFEx
  DISULFID 5..14: note = Disulfide bond between C5 and C14
  VARIANT 1: note = K or K(azido)
  VARIANT 18: note = K or K-SuFEx
XVNSCLLLPNLLGCDGDX

SEQ ID NO: 13 - protein, 17 aa, synthetic construct
  MOD_RES 1: note = K(azido)
  MOD_RES 11: note = K-SuFEx
  DISULFID 5..14: note = Disulfide bond between C5 and C14
  VARIANT 1: note = K or K(azido)
  VARIANT 11: note = K or K-SuFEx
XVNSCLLLPNXLGCGDD

SEQ ID NO: 14 - protein, 17 aa, synthetic construct
  MOD_RES 1: note = K(azido)
  MOD_RES 11: note = K-SuFEx
  DISULFID 5..14: note = Disulfide bond between C5 and C14
  VARIANT 1: note = K or K(azido)
  VARIANT 11: note = K or K-SuFEx
XVNSCLLLPNXLGCDGD

SEQ ID NO: 15 - protein, 14 aa, synthetic construct
  MOD_RES 7: note = X is Cyclohexylalanine
  MOD_RES 9: note = Ser is D-Ser
  MOD_RES 10: note = Arg is D-Arg
KGGSGDXFSRYLWS

SEQ ID NO: 16 - protein, 14 aa, synthetic construct
  MOD_RES 7: note = X is Cyclohexylalanine
  MOD_RES 9: note = Ser is D-Ser
  MOD_RES 10: note = Arg is D-Arg
KGGSGDXFSRALWS

SEQ ID NO: 17 - protein, 19 aa, synthetic construct
  MOD_RES 6..15: note = Disulfide bond between C6 and C15
AQVNSCLLLPNLLGCGDDY